(19) United States
(12) Patent Application Publication
(10) Pub. No.: US 2014/0276229 A1
(43) Pub. Date: Sep. 18, 2014

(54) INFORMATION ANALYZING APPARATUS, DIGITAL STETHOSCOPE, INFORMATION ANALYZING METHOD, MEASUREMENT SYSTEM, CONTROL PROGRAM, AND RECORDING MEDIUM

(71) Applicant: Sharp Kabushiki Kaisha, Osaka-shi, Osaka (JP)

(72) Inventor: Yutaka Ikeda, Osaka-shi (JP)

(21) Appl. No.: 14/353,545

(22) PCT Filed: Dec. 10, 2012
(86) PCT No.: PCT/JP2012/081960

§ 371 Date: Apr. 23, 2014

(30) Foreign Application Priority Data
Dec. 13, 2011 (JP) 2011-272785

(51) Int. Cl. A61B 7/04; A61B 7/00; A61B 19/00; A61B 5/00

(52) U.S. Cl. CPC A61B 7/04; A61B 5/7282; A61B 5/7264; A61B 7/003; A61B 19/5212
USPC 600/586

(57) ABSTRACT

An information analyzing apparatus (100) of the present invention includes a waveform feature determining unit (30) and a sound-type determining unit (40). The waveform feature determining unit (30) applies waveform feature determination criteria used for classifying features of sound waveforms to a sound waveform included in body sound information collected by a stethoscope so as to specify a feature of the sound waveform. The sound-type determining unit (40) determines a sound type to which the body sound information belongs, on the basis of the feature of the sound waveform specified by the waveform feature determining unit (30). With this configuration, it is possible to analyze body sound information objectively and highly precisely and to present analysis results so that a user can efficiently utilize them.

### TECHNICAL FIELD

The present invention relates to an information analyzing apparatus which analyzes body sound information collected by a stethoscope, an information analyzing method, a control program, and a recording medium.

### BACKGROUND ART

Hitherto, digital stethoscopes which collect body sounds (such as respiratory system sounds and heartbeats) from a body (patient or subject person) and record the collected body sounds as digital signals (body sound information) are widely used. By digitally recording body sound information by using a digital stethoscope, a great variety of modes of diagnosis are implemented, which are different from existing modes, for example, a physician examines a patient on a face-to-face basis by using a stethoscope. For example, a physician being in a place away from a patient and an operator of a digital stethoscope is able to receive information concerning collected body sounds and conduct diagnosis in a remote site. Additionally, the use of a digital stethoscope makes it possible for a physician to listen to collected and recorded body sound information later, so that the physician can compare items of information concerning body sounds collected on different dates with each other.

That is, body sound collected by using a stethoscope is not a piece of information that is listened to by a physician only while examining a patient on a face-to-face basis, but a piece of information important for patients that can be recorded and stored in an electronic health record as body sound information. Such body sound information is used, not only for playing back and listening to by a physician, but also for being analyzed by an analyzing apparatus.

For example, PTL 1 discloses a breath-sound-data processing device which analyzes breath sound data. The breath-sound-data processing device checks for adventitious sounds on the basis of sampling data and breath sound data which is actually obtained.

PTL 2 discloses a lung-sound diagnostic device which collects lung sounds and checks for abnormal lung sounds. The lung-sound diagnostic device determines the presence or the absence of abnormal lung sounds by comparison with reference data indicating lung sounds of a certain disease which is already known.

### CITATION LIST

### Patent Literature

PTL 1: Japanese Unexamined Patent Application Publication No. 2005-066044 (publication date: Mar. 17, 2005)
PTL 2: Japanese Unexamined Patent Application Publication No. 2007-190082 (publication date: Aug. 2, 2007)
PTL 3: Japanese Unexamined Patent Application Publication No. 2005-40178 (publication date: Feb. 17, 2005)

### SUMMARY OF INVENTION

### Technical Problem

In a known diagnosis method, body sounds, which are information listened to by a physician only while examining a patient on a face-to-face basis, is utilized in the following manner. A physician listens to body sounds of a patient only while examining the patient on a face-to-face basis and determines the patient's condition from the body sounds on the basis of the expertise and experience of the physician, thereby providing an appropriate diagnosis to the patient. That is, in a diagnosis method depending on the ears of a physician having expertise and experience, it is sufficient even if body sounds are collected and listened to only while a physician is examining a patient.

As stated above, however, through the years, body sound information is recorded as one piece of information concerning patients and is available all the time. Under these circumstances, it can be assumed that body sound information may be utilized in all sorts of diagnostic scenes by users other than a physician actually examining (auscultating) a patient. In this case, users include all sorts of people who may utilize this body sound information, not only physicians, but also health care professionals taking care of the patient other than specialized physicians, or in some cases, all parties related to the patient who do not have medical skills.

Accordingly, by using a known diagnosis method depending on physician's ears, users are unable to obtain necessary information from body sound information and to understand it in a correct manner. Even for users having medical expertise, it takes a considerable time to make correct judgments in order to obtain necessary information by listening to body sound information.

In the techniques disclosed in PTL 1 and PTL 2 of the related art, body sound information is analyzed so that users can be assisted to make correct judgments. In these techniques, however, the presence or the absence of abnormal sounds or adventitious sounds is checked by comparing subject sound data with sampling sound data which is stored in advance (such as normality/abnormality learning data, sampling data, and reference data similar to the subject sound data).

Accordingly, the precision in judging the presence or the absence of abnormal sounds or adventitious sounds depends on the amount of information in a database in which sampling data is stored, thereby making the precision unstable.

It is thus desirable to provide an analyzing apparatus, concerning a known diagnosis method depending on physician's ears, which is capable of objectively and highly precisely analyzing body sound information so that users can be assisted and which is capable of recording or supplying the analyzed body sound information so that users can easily and efficiently utilize it as meaningful information.

The present invention has been made in view of the above-described problems. It is an object of the present invention to implement an information analyzing apparatus which objectively and highly precisely analyzes body sound information collected by a stethoscope and which presents analysis results so that a user can efficiently utilize them, and also to implement an information analyzing method, a control program, and a recording medium.

### Solution to Problem

In order to solve the above-described problems, the present invention provides an information analyzing apparatus including: waveform feature determining means for applying waveform feature determination criteria to a sound waveform included in body sound information collected by a stethoscope so as to specify a feature of the sound waveform, the waveform feature determination criteria indicating criteria for classifying features of sound waveforms; and sound-type determining means for determining a sound type to which the body sound information belongs, on the basis of the feature of the sound waveform specified by the waveform feature determining means.

In order to solve the above-described problems, the present invention provides an information analyzing method including: a waveform feature determining step of applying waveform feature determination criteria to a sound waveform included in body sound information collected by a stethoscope so as to specify a feature of the sound waveform, the waveform feature determination criteria indicating criteria for classifying features of sound waveforms; and a sound-type determining step of determining a sound type to which the body sound information belongs, on the basis of the feature of the sound waveform specified in the waveform feature determining step.

### Advantageous Effects of Invention

In order to solve the above-described problems, the information analyzing apparatus of the present invention includes: waveform feature determining means for applying waveform feature determination criteria to a sound waveform included in body sound information collected by a stethoscope so as to specify a feature of the sound waveform, the waveform feature determination criteria indicating criteria for classifying features of sound waveforms; and sound-type determining means for determining a sound type to which the body sound information belongs, on the basis of the feature of the sound waveform specified by the waveform feature determining means.

In order to solve the above-described problems, the information analyzing method of the present invention includes: a waveform feature determining step of applying waveform feature determination criteria to a sound waveform included in body sound information collected by a stethoscope so as to specify a feature of the sound waveform, the waveform feature determination criteria indicating criteria for classifying features of sound waveforms; and a sound-type determining step of determining a sound type to which the body sound information belongs, on the basis of the feature of the sound waveform specified in the waveform feature determining step.

Accordingly, it is possible to implement an information analyzing apparatus which objectively and highly precisely analyzes body sound information collected by a stethoscope and which presents the analysis results so that a user can efficiently utilize them.

### BRIEF DESCRIPTION OF DRAWINGS

FIG. 1 is a functional block diagram illustrating the major configuration of an information analyzing apparatus according to an embodiment of the present invention.

FIG. 2 illustrates an overview of an auscultation system according to an embodiment of the present invention.

FIG. 3 is a diagram illustrating a specific example of body sound information, in particular, breath sounds of a healthy person, obtained by a body sound obtaining unit of the information analyzing apparatus.

FIG. 4 is a diagram illustrating a specific example of body sound information, in particular, breath sounds of a patient suffering from pneumonia, obtained by the body sound obtaining unit of the information analyzing apparatus.

FIG. 5 shows diagrams illustrating specific examples of autocorrelation functions found by an autocorrelation analyzer of the information analyzing apparatus, more specifically, part (a) is a diagram illustrating an autocorrelation function found by the autocorrelation analyzer by using the waveform of breath sounds shown in FIG. 3 as input, and part (b) is a diagram illustrating another example of an autocorrelation function found by the autocorrelation analyzer by using a waveform of other breath sounds as input.

FIG. 6 is a diagram illustrating a specific example of an autocorrelation function found by the autocorrelation analyzer of the information analyzing apparatus, more specifically, a diagram illustrating an autocorrelation function found by the autocorrelation analyzer by using the waveform of breath sounds shown in FIG. 4 as input.

FIG. 7 is a diagram illustrating examples of waveform feature determination criteria referred to by a periodicity determining section of the information analyzing apparatus and examples of waveform feature determination results output from the periodicity determining section.

FIG. 8 is a diagram illustrating a specific example of a spectrum output from a Fourier transform unit of the information analyzing apparatus, more specifically, a diagram illustrating a spectrum extracted by performing Fourier transform on the breath sounds of a healthy person shown in FIG. 3.

FIG. 9 is a diagram illustrating another specific example of body sound information obtained by the body sound obtaining unit of the information analyzing apparatus, more specifically, a diagram illustrating breath sounds of a patient suffering from asthma.

FIG. 10 is a diagram illustrating a specific example of a spectrum output from the Fourier transform unit of the information analyzing apparatus, more specifically, a diagram illustrating a spectrum extracted by performing Fourier transform on the breath sounds of a patient suffering from asthma shown in FIG. 8.

FIG. 11 is a diagram illustrating examples of waveform feature determination criteria referred to by a spectrum determining section of the information analyzing apparatus and examples of waveform feature determination results output from the spectrum determining section.

FIG. 12 is a diagram illustrating a spectrogram extracted as a result of a time-frequency analyzer of the information analyzing apparatus performing a short-time frequency analysis on breath sounds of a healthy person.

FIG. 13 is a diagram illustrating a spectrogram extracted as a result of the time-frequency analyzer of the information analyzing apparatus performing a short-time frequency analysis on decreased breath sounds.

FIG. 14 is a diagram illustrating a spectrogram extracted as a result of the time-frequency analyzer of the information analyzing apparatus performing a short-time frequency analysis on continuous adventitious sounds.

FIG. 15 is a diagram illustrating a spectrogram extracted as a result of the time-frequency analyzer of the information analyzing apparatus performing a short-time frequency analysis on discontinuous adventitious sounds.

FIG. 16 illustrates examples of waveform feature determination criteria referred to by a spectrogram determining section of the information analyzing apparatus and examples of waveform feature determination results output from the spectrogram determining section.

FIG. 17 is a diagram illustrating a specific example of an envelope of a body sound waveform output from an envelope detector of the information analyzing apparatus.

FIG. 18 is a diagram illustrating examples of waveform feature determination criteria referred to by an envelope determining section of the information analyzing apparatus and examples of waveform feature determination results output from the envelope determining section.

Part (a) of FIG. 19 is a diagram illustrating a specific example of an envelope having a high continuity, and part (b) of FIG. 19 is a diagram illustrating a specific example of an envelope having a low continuity.

FIG. 20 is a diagram illustrating a specific example of impulse noise detection results, in which impulse noise is specified in a sound waveform, output from an impulse noise detector of the information analyzing apparatus.

FIG. 21 is a diagram illustrating examples of waveform feature determination criteria referred to by an impulse noise determining section of the information analyzing apparatus and examples of waveform feature determination results output from the impulse noise determining section.

FIG. 22 is a diagram illustrating a specific example of sound-type determination results which are output from a normal-breath-sound determining section of a sound-type determining unit by using, as input, waveform feature determination results output from a waveform feature determining unit of the information analyzing apparatus.

FIG. 23 is a diagram illustrating a specific example of sound-type determination results which are output from a decreased-breath-sound determining section of the sound-type determining unit by using, as input, waveform feature determination results output from the waveform feature determining unit of the information analyzing apparatus.

FIG. 24 is a diagram illustrating a specific example of sound-type determination results which are output from a continuous-adventitious-sound determining section of the sound-type determining unit by using, as input, waveform feature determination results output from the waveform feature determining unit of the information analyzing apparatus.

FIG. 25 is a diagram illustrating a specific example of sound-type determination results which are output from a discontinuous-adventitious-sound determining section of the sound-type determining unit by using, as input, waveform feature determination results output from the waveform feature determining unit of the information analyzing apparatus.

FIG. 26 is a diagram illustrating examples of decreased-sound-level determination criteria referred to by a decreased-sound-level determining section of the information analyzing apparatus and examples of decreased-sound-level determination results output from the decreased-sound-level determining section.

FIG. 27 is a diagram illustrating examples of continuity-level determination criteria referred to by a continuity-level determining section of the information analyzing apparatus and examples of continuity-level determination results output from the continuity-level determining section.

FIG. 28 is a diagram illustrating examples of discontinuity-level determination criteria referred to by a discontinuity-level determining section of the information analyzing apparatus and examples of discontinuity-level determination results output from the discontinuity-level determining section.

FIG. 29 is a view illustrating a specific example of a display screen for displaying analysis results and level determination results output from a result output unit of the information analyzing apparatus.

FIG. 30 is a flowchart illustrating a flow of information analyzing processing performed by the information analyzing apparatus according to an embodiment of the present invention.

FIG. 31 is a functional block diagram illustrating the major configuration of an information analyzing apparatus according to another embodiment of the present invention.

FIG. 32 is a diagram illustrating a sound type system used by a comprehensive determination section of this embodiment for classifying respiratory system sounds obtained from a patient as a predetermined sound type.

FIG. 33A is a flowchart illustrating a flow of information analyzing processing performed by the information analyzing apparatus of this embodiment.

FIG. 33B is a flowchart illustrating a flow of information analyzing processing performed by the information analyzing apparatus of this embodiment.

FIG. 34 is a flowchart illustrating a flow of decreased-sound-level determining processing performed by a decreased-sound-level determining section of the information analyzing apparatus.

FIG. 35 is a flowchart illustrating a flow of continuity-level determining processing performed by a continuity-level determining section of the information analyzing apparatus.

FIG. 36 is a flowchart illustrating a flow of discontinuity-level determining processing performed by a discontinuity-level determining section of the information analyzing apparatus.

FIG. 37 is a view illustrating another specific example of a display screen for displaying analysis results and level determination results output from a result output unit of the information analyzing apparatus.

FIG. 38 is a block diagram illustrating an overview of a measurement system and the major configuration of an imaging apparatus forming the measurement system.

FIG. 39 is a view illustrating a skeleton of lungs of a body.

### DESCRIPTION OF EMBODIMENTS

### First Embodiment

An embodiment of an information analyzing apparatus of the present invention will be described below with reference to FIGS. 1 through 30.

In the following embodiment, an example in which an information analyzing apparatus of the present invention is applied to an auscultation system will be discussed. The auscultation system is, in this example, a system that implements the following operation. Body sounds of a subject are obtained by using a digital stethoscope, and obtained digital data, that is, body sound information, is analyzed by the information analyzing apparatus of the present invention and is used for medical diagnosis and treatment for the subject. A subject subjected to a medical examination by using a digital stethoscope will be referred to as a “patient”. Although in this embodiment a human being is assumed as a subject (patient), an auscultation system in which all sorts of living bodies other than human beings are assumed as subjects (patients) is also encompassed within the present invention.

In the following description, the information analyzing apparatus of the present invention analyzes respiratory system sounds (body sounds) of a patient and determines the condition of the patient as to pulmonary disease by way of example. However, the information analyzing apparatus of the present invention is not restricted to this example, and may analyze other body sounds (such as heartbeats, abdominal cavity sounds, intestine sounds, blood flow sounds, and fetal heartbeats) and determine the condition of a patient as to a corresponding body part.

The information analyzing apparatus of the present invention is not restricted to the system in the above-described example, and may be applied to all sorts of other systems in which body sound information is obtained from a living body and is utilized for a purpose other than medical diagnosis and treatment.

### [Overview of Auscultation System]

FIG. 2 illustrates an overview of an auscultation system of an embodiment of the present invention. An auscultation system 200 at least includes a digital stethoscope 3 used for collecting (auscultating) body sounds from a patient P by an operator U, and an information analyzing apparatus 100 used by the operator U when auscultating body sounds.

The operator U is in a clinic 1 where medical diagnosis and treatment is given to the patient P, and checks the patient P in the clinic 1 by using various devices, such as the digital stethoscope 3. In this case, the various devices may include an oximeter, an electrocardiograph, a sphygmomanometer, a thermometer, an arteriosclerosis meter, and a blood vessel aging measuring device.

The information analyzing apparatus 100 and the digital stethoscope 3 are connected to each other so that they can communicate with each other via a wired or wireless medium. By operating the information analyzing apparatus 100, the operator U is able to read and refer to information necessary for examining the patient P, for example, information concerning the patient P (electronic health record). The operator U is also able to store body sound information collected from the digital stethoscope 3 in the information analyzing apparatus 100.

The information analyzing apparatus 100 is implemented by an information processing terminal having a high portability owned by the operator U, or a desk-top personal computer (PC) installed in the clinic 1. In the example shown in FIG. 2, the information analyzing apparatus 100 of the present invention is implemented by a multifunction mobile communication terminal, such as a smartphone, by way of example.

If the operator U has medical expertise, skills, and authority as a physician, he/she may examine the patient P by using the digital stethoscope 3 and the information analyzing apparatus 100, and may give treatment to the patient P by making a final judgment of the condition of the patient P. In this manner, the auscultation system 200 including the digital stethoscope 3 and the information analyzing apparatus 100 is also encompassed within the present invention.

Alternatively, as shown in FIG. 2, the auscultation system 200 may be constructed by including the digital stethoscope 3 and the information analyzing apparatus 100 in the clinic 1 and also including a management server 4 in a support center 2 of a remote site. In this case, the information analyzing apparatus 100 and the management server 4 are connected to each other so that they can communicate with each other via a communication network 5, such as the Internet.

More specifically, the following situation may be considered. The operator U may have skills to operate the digital stethoscope 3 and the information analyzing apparatus 100 and to perform simple medical checking and treatment on the spot in the clinic 1 under the guidance of a specialized physician, though the operator U does not have the same levels of expertise, skills, and authority as those of the physician or the operator U is not a specialist of the field of currently conducted medical checking and treatment. Under this situation, the digital stethoscope 3 and the information analyzing apparatus 100 operated by the operator U, such as a nurse practitioner (NP) or another health care professional, are disposed in the clinic 1 of the auscultation system 200, and in the support center 2 located away from the clinic 1, the management server 4 which manages electronic health records of individual patients in the auscultation system 200 is disposed. A physician D having special expertise and skills stays in the support center 2, and gives guidance to the operator U by using a communication device (not shown), such as an information processing terminal or a telephone, so as to assist the operator U to conduct diagnosis and treatment. Meanwhile, body sound information directly collected from the patient P by the operator U by using the digital stethoscope 3 is stored in the management server 4 via the information analyzing apparatus 100. The physician D is able to give instructions concerning diagnosis and treatment by accessing the management server 4 and obtaining body sound information concerning the patient P being in a remote site. Under the guidance of the physician D, the operator U is able to conduct simple treatment, or if it is difficult to handle this patient P in the clinic 1, the operator U is able to introduce a hospital, which may give a suitable treatment, cooperated with this clinic 1.

In this embodiment, the information analyzing apparatus 100 implemented by a smartphone has a function of analyzing body sound information collected from the digital stethoscope 3 and outputting analysis results to the information analyzing apparatus 100 or the management server 4. The information analyzing apparatus 100 of the present invention having a function of analyzing body sound information may be implemented as the management server 4 in a remote site.

The configuration and the operation of this information analyzing apparatus 100 will be described below in detail.

### [Hardware Configuration of Information Analyzing Apparatus]

FIG. 1 is a functional block diagram illustrating the major configuration of the information analyzing apparatus 100 of this embodiment.

As the hardware configuration, the information analyzing apparatus 100 at least includes a controller 10, an input unit 11, a display unit 12, a storage unit 13, and a communication unit 14. For implementing regular functions of a smartphone, the information analyzing apparatus 100 may include various regular components of a smartphone, such as a sound input unit, an external interface, a sound output unit, a speech communication processor, a broadcasting receiver (such as a tuner and a demodulator), a GPS, sensors (such as an acceleration sensor and an orientation sensor), and an imaging unit.

In this embodiment, since the information analyzing apparatus 100 is a smartphone, the input unit 11 and the display unit 12 are integrally formed as a touch panel. If the information analyzing apparatus 100 is implemented by, for example, a PC, the display unit 12 may be, for example, a liquid crystal display monitor, and the input unit 11 may be, for example, a keyboard and a mouse.

The input unit 11 is used for allowing a user to input an instruction signal to operate the information analyzing apparatus 100 via the touch panel. The input unit 11 is constituted by a touch face and a touch sensor. The touch face receives contact of a pointer (such as a finger or a pen). The touch sensor detects contact/non-contact (access/non-access) between a pointer and the touch face and also detects a contact (access) position. The touch sensor may be implemented by any type of sensor, for example, a pressure sensor, an electrostatic capacitive sensor, an optical sensor, as long as it is able to detect contact/non-contact between a pointer and the touch panel.

The display unit 12 displays results of processing body sound information by the information analyzing apparatus 100 and also displays an operation screen for allowing a user to operate the information analyzing apparatus 100 as a GUI (Graphical User Interface) screen. The display unit 12 is implemented by, for example, an LCD (liquid crystal display).

The information analyzing apparatus 100 may include, in addition to the input unit 11, an operation unit (not shown) for allowing a user to directly input an instruction signal into the information analyzing apparatus 100. For example, the operation unit is implemented by a suitable input mechanism, such as a button, a switch, a key, and a jog dial. The operation unit may be a switch for turning ON/OFF the power of the information analyzing apparatus 100.

The communication unit 14 communicates with external devices (such as the digital stethoscope 3 and the management server 4). In this embodiment, the communication unit 14 includes a near-field communication section for performing near-field communication with the digital stethoscope 3. The near-field communication section performs wireless communication with the digital stethoscope 3 and receives, from the digital stethoscope 3, body sound information obtained by digitizing body sounds collected by the digital stethoscope 3. The type of near-field communication section is not particularly restricted, and may implement one or a plurality of wireless communication means such as infrared communication, such as IrDA or IrSS, Bluetooth (registered) communication, WiFi communication, a non-contact IC card.

The communication unit 14 may include a remote communication section which performs data communication with a device (such as the management server 4) located in a remote site via the communication network 5 (such as a LAN (Local Area Network) or a WAN (Wide Area Network)). The remote communication section is able to send, for example, results of analyzing body sound information by the information analyzing apparatus 100 to the management server 4 via the communication network 5.

If the information analyzing apparatus 100 is a cellular phone, such as a smartphone, the communication unit 14 may have a function of sending and receiving voice communication data, email data, and so on, to and from other devices via a cellular phone circuit network.

The storage unit 13 is a device that stores (1) a control program executed by the controller 10 of the information analyzing apparatus 100, (2) an OS program executed by the controller 10, (3) application programs for executing various functions of the information analyzing apparatus 100 by the controller 10, and (4) various items of data which are read when these application programs are executed. Alternatively, the storage unit 13 is a device that stores (5) data used for calculations while the controller 10 is executing various functions and calculation results. The above-described items of data (1) through (4) are stored in a non-volatile storage device, such as a ROM (read only memory), a flash memory, an EPROM (Erasable Programmable ROM), an EEPROM (registered trademark) (Electrically EPROM), or an HDD (Hard Disk Drive). The above-described item of data (5) is stored in a volatile storage device, such as a RAM (Random Access Memory). Decisions concerning which item of data will be stored in which storage device are suitably made by considering the purpose of use of the information analyzing apparatus 100, convenience, costs, physical restrictions. For example, collected sound body information concerning a patient P is temporarily stored in the RAM and is then read by the controller 10 of the information analyzing apparatus 100. Results of analyzing body sound information by the controller 10 (and body sound information if necessary) are stored in the storage unit 13 implemented by a non-volatile storage device, such as a ROM.

The controller 10 centrally controls individual elements included in the information analyzing apparatus 100. The controller 10 is implemented by, for example, a CPU (central processing unit). Functions of the information analyzing apparatus 100 are implemented by reading a program stored in, for example, a ROM into, for example, a RAM by the controller 10, which serves as a CPU. Various functions (in particular, an information analyzing function) implemented by the controller 10 will be discussed later in detail with reference to drawings different from FIG. 1.

### [Functional Configuration of Information Analyzing Apparatus]

As shown in FIG. 1, the controller 10 of the information analyzing apparatus 100 includes, as functional blocks, a body sound obtaining unit 20, a body sound processor 21, a body sound analyzer 22, and a result output unit 23.

The body sound obtaining unit 20 obtains body sound information concerning a patient P received by the communication unit 14 from the digital stethoscope 3. The body sound obtaining unit 20 temporarily stores received body sound information in the storage unit 13, and reads it when necessary and supplies it to elements (such as the body sound processor 21) on a downstream side.

The body sound processor 21 processes sound waveforms indicated by body sound information obtained by the body sound obtaining unit 20 and extracts waveform feature information concerning the sound waveforms. Waveform feature information is obtained by plotting sound waveforms contained in the body sound information on a two-dimensional graph or a three or more dimensional graph, by using, as indexes, various information concerning the sound waveforms or individual sound components forming the sound waveforms. Examples of various information concerning sound components are the frequency, amplitude values, and generation times, but various information concerning sound components is not restricted to these examples. In this manner, by extracting waveform feature information generated by the body sound processor 21, features of sound waveforms can be digitized in terms of various viewpoints by using various indexes and can be simply handled as features quantities. Extracted waveform feature information and feature quantities calculated from the waveform feature information are utilized for analyzing sound waveforms by the body sound analyzer 22.

In this embodiment, the body sound processor 21 is implemented by at least one of an autocorrelation analyzer 211, a Fourier transform unit 212, a time-frequency analyzer 213, an envelope detector 214, and an impulse noise detector 215, though it is not restricted thereto. These elements of the body sound processor 21 extract waveform feature information concerning the functions of the corresponding elements. Details of the individual elements will be discussed later.

The body sound analyzer 22 determines, on the basis of waveform feature information concerning body sounds extracted by the body sound processor 21, the condition of a patient who has emitted these body sounds. More specifically, in this embodiment, the body sound analyzer 22 includes at least a waveform feature determining unit 30 and a sound-type determining unit 40. The body sound analyzer 22 may preferably also include an abnormality-level determining unit 50.

The waveform feature determining unit 30 determines whether extracted waveform feature information matches waveform feature criteria, and then classifies and specifies the features of sound waveforms indicated by the waveform feature information. The waveform feature determining unit 30 may determine whether or not one item of waveform feature information matches each of a plurality of waveform feature criteria. Alternatively, the waveform feature determining unit 30 may determine whether or not each of a plurality of items of waveform feature information extracted from one sound wave matches each of a plurality of waveform feature criteria. The waveform feature criteria are defined and stored in the storage unit 13 in advance. The waveform feature determining unit 30 reads the waveform feature criteria stored in the storage unit 13 and determines whether or not extracted waveform feature information matches the waveform feature criteria. This makes it possible to clearly classify the types of features of a sound waveform indicated by the waveform feature information. Information concerning the sound waveform for which features are classified by the waveform feature determining unit 30 in this manner is output to the sound-type determining unit 40 as waveform feature determination results. The waveform feature determination results are used for determining a sound type of sound waveform by the sound-type determining unit 40.

In this embodiment, the waveform feature determining unit 30 is implemented by at least one of a periodicity determining section 31, a spectrum determining section 32, a spectrogram determining section 33, an envelope determining section 34, and an impulse noise determining section 35, though it is not restricted thereto. Details of the individual elements will be discussed later.

The sound-type determining unit 40 determines, on the basis of waveform feature determination results output from the waveform feature determining unit 30, a sound type of body sound information indicated by a sound waveform in the waveform feature determination results. In this embodiment, the sound type is a type of sound obtained by classifying sounds contained in body sound information collected from a patient on the basis of medical features. That is, the sound-type determining unit 40 serves as means for classifying sounds contained in collected body sound information on the basis of medical features by determining the types of body sound information.

In this manner, by using the waveform feature determining unit 30 and the sound-type determining unit 40, respiratory system sounds of a patient are classified as a certain type of sound on the basis of medical features. Accordingly, the body sound analyzer 22 is able to determine the condition (illness) of a patient who has emitted the classified type of respiratory system sound.

In this embodiment, the information analyzing apparatus 100 is a device for analyzing, as body sounds, respiratory system sounds. Accordingly, the sound-type determining unit 40 may classify respiratory system sounds, for example, as the following sound types, on the basis of medical features.

For example, the sound-type determining unit 40 may classify collected body sounds as “breath sounds (sounds accompanied by expiration and sounds accompanied by inspiration)” and “adventitious sounds (sounds other than expiration sounds and inspiration sounds, generated by a disease)”. The sound-type determining unit 40 may also classify “breath sounds” as “normal breath sounds” and “abnormal breath sounds”. The sound-type determining unit 40 may also classify “abnormal breath sounds” as “decreased (absent) breath sounds”, “increased breath sounds”, “prolonged expiration”, “bronchial breath sounds”, and “windpipe stridor sounds”. The sound-type determining unit 40 may also classify “adventitious sounds” as “continuous adventitious sounds”, “discontinuous adventitious sounds”, “pleural friction rub”, and “pulmonary vascular adventitious sounds”. The sound-type determining unit 40 may also classify “continuous adventitious sounds” as “high-pitched continuous adventitious sounds” and “low-pitched adventitious sounds”. The sound-type determining unit 40 may classify “discontinuous adventitious sounds” as “fine discontinuous adventitious sounds” and “coarse discontinuous adventitious sounds”.

Alternatively, the sound-type determining unit 40 may make a determination whether or not breath sounds are applied to a certain type of sound and then return a binary value indicating, for example, whether breath sounds are normal breath sounds or there is a possibility that breath sounds are not normal breath sounds.

A mechanism in which decreased breath sounds are generated is as follows. A case in which an obstacle, such as pleural effusion, is stored between lungs and a thoracic cavity may be considered. If an obstacle exists in a path from lungs in which normal breath sounds are generated until a stethoscope, this obstacle serves as a so-called low-pass filter and cuts high frequency components. A case in which an obstacle exists between lungs and a thoracic cavity is frequently observed among patients suffering from pleural effusion, pneumothorax, atelectasis, or pulmonary emphysema. Accordingly, if the information analyzing apparatus 100 of the present invention classifies body sounds as decreased breath sounds, the operator U or the physician D may be able to diagnose the disease of a patient as pleural effusion, pneumothorax, atelectasis, or pulmonary emphysema.

A mechanism in which continuous adventitious sounds are generated is as follows. If secretion is stored in a trachea, the flow of expiration or inspiration air flowing within the trachea is disturbed, thereby emitting adventitious sounds. Then, these adventitious sounds are continuously emitted all through while expiration or inspiration air is flowing. The storage of secretion is frequently observed among patients suffering from asthma, obstructive lung disease (such as pulmonary emphysema and chronic obstructive pulmonary disease), and tracheal stenosis and bronchial stenosis. Accordingly, if the information analyzing apparatus 100 of the present invention classifies body sounds as continuous adventitious sounds, the operator U or the physician D may be able to diagnose the disease of a patient as asthma, obstructive lung disease (such as pulmonary emphysema and chronic obstructive pulmonary disease), or tracheal stenosis and bronchial stenosis.

The frequency of sound emitted in a thin portion of the respiratory tract (smaller-diameter portion of the tracheal), that is, the lower part of lungs (or a deeper level of the tracheal branched off from the upper part of the tracheal) is high. This sound type can be classified as high-pitched continuous adventitious sounds. On the other hand, the frequency of sound emitted in a thick portion of the respiratory tract (larger-diameter portion of the tracheal), that is, the upper part of lungs (or a shallower level of the tracheal branched off from the upper part of the tracheal) is low. This sound type can be classified as low-pitched continuous adventitious sounds. Accordingly, if the information analyzing apparatus 100 of the present invention classifies body sounds as high-pitched continuous adventitious sounds or low-pitched continuous adventitious sounds, the operator U or the physician D may be able to determine in which part (the upper or lower part) of the lungs the abnormal continuous adventitious sounds are being emitted.

A mechanism in which discontinuous adventitious sounds are generated is as follows. It may be possible that liquid secretion in a trachea form a thin liquid film in the trachea and block the respiratory tract. In this case, if expiration and inspiration air flows within the trachea, the sound of bursting the film is generated. Such a film is formed in places of the trachea, and only when such a film is broken, is bursting sound instantaneously generated. In terms of this point, a type of sound definitely different from continuous adventitious sounds is generated. The above-described storage of liquid secretion is frequently observed among patients suffering from pneumonia.

Accordingly, if the information analyzing apparatus 100 of the present invention classifies body sounds as discontinuous adventitious sounds, the operator U or the physician D may be able to diagnose the disease of a patient as pneumonia.

In a thinner portion of the respiratory tract, a film having a smaller diameter is formed, and such a film is easily broken. Accordingly, the period for which sound is emitted is relatively short. This type of sound can be classified as fine discontinuous adventitious sounds. On the other hand, in a thicker portion of the respiratory tract, a film having a larger diameter is formed, and it takes slightly more time to cause a film to be broken than a film having a smaller diameter. Accordingly, the period for which sound is emitted is relatively long. This type of sound can be classified as coarse discontinuous adventitious sounds. Thus, if the information analyzing apparatus 100 of the present invention classifies body sounds as fine discontinuous adventitious sounds or coarse discontinuous adventitious sounds, the operator U or the physician D may be able to determine in which part (the upper or lower part) of the lungs the abnormal discontinuous adventitious sounds are being emitted.

In this embodiment, the sound-type determining unit 40 is implemented by at least one of a normal-breath-sound determining section 41, a decreased-breath-sound determining section 42, a continuous-adventitious-sound determining section 43, and a discontinuous-adventitious-sound determining section 44, though it is not restricted thereto. Details of the individual elements will be discussed later.

Sound-type determination results obtained by the sound-type determining unit 40 are supplied to the result output unit 23 or are stored in the storage unit 13.

Concerning a sound waveform classified as a specific type, the abnormality-level determining unit 50 determines the degree (level) of this type of sound waveform on the basis of extracted waveform feature information. In particular, the abnormality-level determining unit 50 determines the abnormality degree (such as disease seriousness and progression levels) of abnormal sound types.

In this embodiment, the abnormality-level determining unit 50 determines the abnormality level by determining whether or not extracted waveform feature information matches determination criteria. That is, the abnormality-level determining unit 50 compares extracted waveform feature information with each of the level determination criteria having different thresholds in a stepwise manner, and determines which level determination criterion the waveform feature information matches, thereby determining the abnormality level of body sounds. The level determination criteria are defined and stored in the storage unit 13 in advance.

For example, the abnormality-level determining unit 50 may determine that the abnormality level of body sounds having a relatively high (serious) degree of abnormality is “high”, and the abnormality-level determining unit 50 may determine that the abnormality level of body sounds having a relatively low (mild) degree of abnormality is “low”. The abnormality-level determining unit 50 may determine that the abnormality level of body sounds having a degree of abnormality which is between the high degree and the low degree is “intermediate”.

In this embodiment, the abnormality-level determining unit 50 is implemented by at least one of a decreased-sound-level determining section 51, a continuity-level determining section 52, and a discontinuity-level determining section 53. Details of the individual elements will be discussed later.

Level determination results obtained by the abnormality-level determining unit 50 are supplied to the result output unit 23 or are stored in the storage unit 13.

The result output unit 23 is a unit which outputs sound-type determination results output from the sound-type determining unit 40 as analysis results of analyzing body sound information. If the controller 10 includes the abnormality-level determining unit 50, the result output unit 23 outputs analysis results by including level determination results output from the abnormality-level determining unit 50 in the analysis results. The analysis results output from the result output unit 23 are supplied to the display unit 12 as a video signal and are displayed in the display unit 12 so that the operator U can visually recognize the analysis results.

With the above-described configuration, the body sound processor 21 processes body sound information and extracts waveform feature information from a sound waveform, and the waveform feature determining unit 30 determines which determination criterion the waveform feature information matches (or does not match). The sound-type determining unit 40 is able to determine the type of sound in accordance with the waveform feature determination results. Sound-type determination results obtained by the sound-type determining unit 40 are displayed in the display unit 12 as analysis results.

Concerning the above-described determination criteria, thresholds are defined in advance on the basis of medical features highly related to each sound type. Accordingly, depending on whether or not extracted waveform feature information matches the determination criteria, the sound-type determining unit 40 is able to determine with which sound type the original body sound information has a high correlation.

With this configuration, the type of body sound information can be specified without directly comparing it with model waveforms. Accordingly, it is possible to implement an information analyzing apparatus that highly precisely and efficiently conducts objective analyses without depending on the completeness of a model sound waveform database and that provides analysis results to a user.

The functional blocks of the above-described controller 10 are implemented as a result of, for example, a CPU (central processing unit), reading a program stored in a storage device (storage unit 13) implemented by, for example, a ROM (read only memory) or an NVRAM (non-volatile random access memory) into, for example, a RAM (random access memory), and executing the read program.

### [Details of Functional Configuration of Information Analyzing Apparatus]

A detailed description will first be given of the body sound processor 20 and the waveform feature determining unit 30.

### (Periodicity Determining Function)

FIGS. 3 and 4 are diagrams illustrating specific examples of body sound information obtained by the body sound obtaining unit 20.

Parts (a) and (b) of FIG. 5 and FIG. 6 are diagrams illustrating specific examples of autocorrelation functions output from the autocorrelation analyzer 211.

The autocorrelation analyzer 211 of the body sound processor 21 analyzes a sound waveform included in body sound information obtained by the body sound obtaining unit 20 so as to find an autocorrelation function.

The periodicity determining section 31 of the waveform feature determining unit 30 applies waveform feature determination criteria to the autocorrelation function output from the autocorrelation analyzer 211 so as to determine features (in particular, the periodicity) of a sound waveform having this autocorrelation function.

In the case of normal body sounds (breath sounds) collected from a healthy person, the sound waveform can be assumed as a periodic signal in which expiration and inspiration forms one period, since a healthy person breathes in a stable manner. The autocorrelation analyzer 211 serves as means for analyzing this periodic signal. Autocorrelation is an index for evaluating the correlation between a certain signal v(t) and a signal v(t+τ) obtained by shifting this certain signal by using a time lag, and can be expressed by the following equation as a function R(τ) having the time lag τ as a variable.

$$R ( τ ) = {lim}_{T \to ∞} \frac{1}{T} {\int }_{0}^{T} v ( t ) \cdot v ( t + τ ) d t \qquad [ Math . 1 ]$$

The autocorrelation analyzer 211 supplies the found autocorrelation function to the periodicity determining section 31 as waveform feature information.

FIG. 3 is a diagram illustrating breath sounds of a healthy person. Part (a) of FIG. 5 is a diagram illustrating an autocorrelation function found by the autocorrelation analyzer 211 by using the waveform of the breath sounds shown in FIG. 3 as input. Part (b) of FIG. 5 is a diagram illustrating another example of an autocorrelation function found by the autocorrelation analyzer 211 by using the waveform of other breath sounds as input. In the examples shown in parts (a) and (b) of FIG. 5, the autocorrelation function on the vertical axis is standardized with respect to the peak amplitude.

Upon receiving the autocorrelation function (waveform feature information) shown in part (a) of FIG. 5, the periodicity determining section 31 determines whether or not the autocorrelation function matches waveform feature determination criteria.

The periodicity determining section 31 first determines, on the basis of the autocorrelation function, the strength or the weakness of the periodicity of the sound waveform, and if the periodicity is found, the length of one period (feature quantity).

More specifically, from the autocorrelation function shown in part (a) of FIG. 5, the periodicity determining section 31 detects peaks at intervals of about three seconds and determines that there is a periodicity in which one period has about three seconds. Alternatively, from the autocorrelation function shown in part (b) of FIG. 5, the periodicity determining section 31 detects peaks at intervals of about two seconds and determines that there is a periodicity in which one period has about two seconds.

In this case, the periodicity determining section 31 may determine the degree of the strength of the periodicity in accordance with the ratio of the peaks of the autocorrelation to the autocorrelation other than the peaks (as the periodicity is stronger, the ratio is greater). For example, the periodicity determining section 31 may find a peak width (duration) with respect to the amplitude value at a position of ¼ of a peak amplitude value of the envelope of the autocorrelation function and determine the proportion of this peak width to the breathing period. As this value (feature quantity) is smaller, the periodicity is stronger.

FIG. 4 is a diagram illustrating breath sounds of a patient suffering from pneumonia. FIG. 6 is a diagram illustrating an autocorrelation function found by the autocorrelation analyzer 211 by using the waveform of the breath sounds shown in FIG. 4 as input. In the example shown in FIG. 6, the autocorrelation function on the vertical axis is standardized with respect to the peak amplitude.

In the example shown in FIG. 6, since adventitious sounds other than expiration and inspiration sounds are generated, the autocorrelation is low, and no strong periodicity can be found in the sound waveform. Accordingly, when such an autocorrelation function is input, the periodicity determining section 31 determines that the periodicity of the sound waveform is weak.

As discussed above, by using an autocorrelation function output from the autocorrelation analyzer 211, the periodicity determining section 31 is able to evaluate a sound waveform of body sounds of a subject person as to whether the periodicity is strong or weak, and more specifically, how much the periodicity is strong (weak).

Then, the periodicity determining section 31 reads waveform feature determination criteria stored in the storage unit 13, and applies them to the autocorrelation function. The periodicity determining section 31 then determines whether the features of the autocorrelation function (in this case, the strength of the periodicity and the length of a period) match the waveform feature determination criteria. With this operation, the periodicity determining section 31 is able to specify features of the sound waveform having this autocorrelation function in terms of the periodicity.

FIG. 7 illustrates examples of waveform feature determination criteria referred to by the periodicity determining section 31 and examples of waveform feature determination results output from the periodicity determining section 31.

In this embodiment, the periodicity determining section 31 executes determination item 1 or determination item 1′ in accordance with the waveform feature determination criteria shown in FIG. 7 and outputs waveform feature determination results. The periodicity determining section 31 outputs a binary value, that is, true or false, concerning each of the determination items, as waveform feature determination results.

However, the content shown in FIG. 7 is only an example for explaining the functions of the periodicity determining section 31, and it is not intended to restrict the configuration of the periodicity determining section 31. Thresholds (values between “**_” and “_**”) defined in the waveform feature determination criteria shown in FIG. 7 may be changed and set as desired by a user (such as the operator U) of the information analyzing apparatus 100. Instead of using binary values, that is, true or false, the periodicity determining section 31 may output waveform feature determination results with more details than binary values.

### (Determination Item 1: Determining Whether or not the Periodicity is Strong)

The periodicity determining section 31 executes determination item 1 shown in FIG. 7 so as to determine the strength or the weakness of the periodicity of a body sound waveform. Concerning determination item 1, if the periodicity is strong, the periodicity determining section 31 returns “true”, and if the periodicity is weak, the periodicity determining section 31 returns “false”.

In this embodiment, first, the periodicity determining section 31 executes determination item 1-1 of determination item 1. That is, the periodicity determining section 31 determines whether the waveform of an autocorrelation function has peaks at intervals of two to five seconds. If peaks at intervals of two to five seconds are detected, the periodicity determining section 31 returns “true”, and if peaks at intervals of two to five seconds are not detected, the periodicity determining section 31 returns “false”.

Then, the periodicity determining section 31 executes determination item 1-2. That is, the periodicity determining section 31 determines whether a peak width (horizontal axis; time) with respect to the amplitude value at a position of ¼ of a peak amplitude value (vertical axis) in the envelope of the autocorrelation function is 10% or smaller of the breathing period. If the peak width is 10% or smaller (if the periodicity is strong), the periodicity determining section 31 returns “true”, and if the peak width is greater than 10% (if the periodicity is weak), the periodicity determining section 31 returns “false”.

For example, it is assumed that the period of an autocorrelation function is five seconds and that the average of multiple peak amplitude values observed in the envelope of the autocorrelation function is 0.8. In this case, if the average of the peak widths with respect to the amplitude value of 0.2 in the envelope is 0.5 seconds or smaller, the periodicity determining section 31 determines determination item 1-2 to be true.

Finally, the periodicity determining section 31 integrates the results of determination item 1-1 and determination item 1-2 and outputs the waveform feature determination results of determination item 1. In the example shown in FIG. 7, if both of determination item 1-1 and determination item 1-2 are true, the periodicity determining section 31 determines determination item 1 to be true (that is, the periodicity is strong). If the determination results are other cases, that is, if at least one of determination item 1-1 and determination item 1-2 is false, the periodicity determining section 31 determines determination item 1 to be false (that is, the periodicity is weak).

### (Determination Item 1′: Determining Whether or not the Periodicity is Weak)

When executing determination item 1′, the periodicity determining section 31 also executes determination item 1-1 and determination item 1-2, in a manner similar to the above-described determination item 1. However, in determination item 1′, an approach to integrating the results of determination item 1-1 and determination item 1-2 is different from that of determination item 1.

In the example shown in FIG. 7, if at least one of determination item 1-1 and determination item 1-2 is false, the periodicity determining section 31 determines determination item 1′ to be true (that is, the periodicity is weak). If the determination results are other cases, that is, if both of determination item 1-1 and determination item 1-2 are true, the periodicity determining section 31 determines determination item 1′ to be false (that is, the periodicity is strong).

The periodicity determining section 31 outputs “true” or “false” concerning determination item 1 or determination item 1′ to the sound-type determining unit 40 as waveform feature determination results.

### (Feature Determining Function Based on Frequency Component Distribution)

FIG. 9 is a diagram illustrating another specific example of body sound information obtained by the body sound obtaining unit 20.

FIGS. 8 and 10 are diagrams illustrating specific examples of spectra output from the Fourier transform unit 212.

The Fourier transform unit 212 of the body sound processor 21 analyzes a sound waveform included in body sound information obtained by the body sound obtaining unit 20 so as to extract a spectrum.

The spectrum determining section 32 of the waveform feature determining unit 30 applies waveform feature determination criteria to a spectrum output from the Fourier transform unit 212 so as to determine features of the spectrum (in particular, features concerning frequency components). More specifically, the spectrum determining section 32 determines whether the frequency component distribution in the spectrum indicates that the sound waveform is likely to be normal or abnormal (containing adventitious sounds).

Body sounds are constituted by various frequency components ranging from nearly a direct current (0 Hz) to higher than 1000 Hz. Information concerning the frequency components varies depending on, for example, the presence or the absence of a disease, and if any, the type of disease and the degree of disease. For handling the frequency component information, in this embodiment, the Fourier transform unit 212 performs Fourier analysis. The Fourier transform unit 212 supplies a spectrum extracted from a sound waveform to the spectrum determining section 32 as waveform feature information.

FIG. 8 is a diagram illustrating a spectrum extracted as a result of the Fourier transform unit 212 performing Fourier transform on the breath sounds of a healthy person shown in FIG. 3.

FIG. 9 is a diagram illustrating breath sounds of a patient suffering from asthma.

FIG. 10 is a diagram illustrating a spectrum extracted as a result of the Fourier transform unit 212 performing Fourier transform on the breath sounds of a patient suffering from asthma shown in FIG. 8. FIGS. 8 and 10 show spectra obtained by performing Fourier transform on sound components of body sound waveforms collected for predetermined seconds (for example, 20 seconds).

As shown in FIG. 8, for example, in the case of breath sounds of a healthy person, most (about 80% or higher) of the signal components are positioned at 200 Hz or lower. In contrast, in the case of breath sounds of a patient suffering from asthma, as shown in FIG. 10, many signal components are positioned in a band from 300 to 400 Hz. This is a symptom appearing as a result of a respiratory tract vibrating in a high frequency range since a narrow segment within the respiratory tract has disturbed the air flow. In this manner, by utilizing Fourier analysis performed by the Fourier transform unit 212, the spectrum determining section 32 is able to determine the presence or the absence of a disease (for example, the possibility of asthma) or whether or not adventitious sounds have been generated.

In this embodiment, the spectrum determining section 32 reads waveform feature determination criteria stored in the storage unit 13 and applies them to the above-described spectrum. Then, the spectrum determining section 32 determines whether or not the spectrum matches the waveform feature determination criteria. More specifically, for example, the spectrum determining section 32 calculates, from the spectrum, the proportion of signal components positioned at 200 Hz or lower to all signal components as a feature quantity, and compares this feature quantity with thresholds included in the waveform feature determination criteria.

With this operation, the spectrum determining section 32 is able to classify and specify features of the sound waveform having this spectrum in terms of the frequency components.

FIG. 11 illustrates examples of waveform feature determination criteria referred to by the spectrum determining section 32 and examples of waveform feature determination results output from the spectrum determining section 32.

In this embodiment, the spectrum determining section 32 executes determination item 2-A or determination item 2-B in accordance with the waveform feature determination criteria shown in FIG. 11 and outputs waveform feature determination results. The spectrum determining section 32 outputs a binary value, that is, true or false, concerning each of the determination items, as waveform feature determination results.

However, the content shown in FIG. 11 is only an example for explaining the functions of the spectrum determining section 32, and it is not intended to restrict the configuration of the spectrum determining section 32. Thresholds (values between “**_” and “_**”) defined in the waveform feature determination criteria shown in FIG. 11 may be changed and set as desired by a user (such as the operator U) of the information analyzing apparatus 100. Instead of using binary values, that is, true or false, the spectrum determining section 32 may output waveform feature determination results with more details than binary values.

(Determination Item 2-A: Determining Whether or not Frequency Component Distribution Indicates that Body Sound Information is Likely to be Normal)

The spectrum determining section 32 executes determination item 2-A shown in FIG. 11. In determination item 2-A, the spectrum determining section 32 determines whether or not the total frequency components at 200 Hz or lower occupies 80% or higher of all frequency components. As shown in FIG. 8, if the total frequency components at 200 Hz or lower occupies 80% or higher of all frequency components, it can be assumed that the body sound information is likely to be normal. In determination item 2-A, determinations are made as follows. If the total frequency components at 200 Hz or lower occupies 80% or higher of all frequency components, the spectrum determining section 32 outputs “true (substantially normal)” to the sound-type determining unit 40 as waveform feature determination results. If the total frequency components at 200 Hz or lower occupies smaller than 80% of all frequency components, the spectrum determining section 32 outputs “false (may not be normal)” to the sound-type determining unit 40 as waveform feature determination results.

(Determination Item 2-B: Determining Whether or not Frequency Component Distribution Indicates that Body Sound Information is Likely to be Abnormal)

The spectrum determining section 32 executes determination item 2-B shown in FIG. 11. In determination item 2-B, the spectrum determining section 32 determines whether or not the total frequency components at 200 Hz or higher occupies 30% or higher of all frequency components. As shown in FIG. 10, if many frequency components at 200 Hz or higher are observed, there may be a sign of abnormality. In determination item 2-B, determinations are made as follows. If the total frequency components at 200 Hz or higher occupies 30% or higher of all frequency components, the spectrum determining section 32 outputs “true (there is a sign of abnormality)” to the sound-type determining unit 40 as waveform feature determination results. If the total frequency components at 200 Hz or higher occupies smaller than 30% of all frequency components, the spectrum determining section 32 outputs “false (there is no sign of abnormality)” to the sound-type determining unit 40 as waveform feature determination results.

### (Feature Determining Function Based on Time-Frequency Components)

FIGS. 12 through 15 are diagrams illustrating specific examples of spectrograms output from the time-frequency analyzer 213.

The time-frequency analyzer 213 of the body sound processor 21 analyzes a sound waveform included in body sound information obtained by the body sound obtaining unit 20 by a predetermined unit time so as to find a spectrogram.

The spectrogram determining section 33 of the waveform feature determining unit 30 applies waveform feature determination criteria to a spectrogram output from the time-frequency analyzer 213 so as to determine features of the spectrogram. More specifically, the spectrogram determining section 33 specifies a frequency having a periodicity (or not having a periodicity) as a feature quantity or determines the strength or the weakness of the periodicity in each frequency range.

A spectrum output from the Fourier transform unit 212 is a two-dimensional graph having frequency components (intensity) on the vertical axis and a frequency on the horizontal axis. Since time information is missing in the spectrum, it is not possible to observe how the frequency components in each frequency range change over time.

In contrast, a spectrogram output from the time-frequency analyzer 213 is a three-dimensional graph to which time information is added. For example, a spectrogram may be created as follows. The frequency components indicated by colors are plotted on a two-dimensional graph having a frequency on the vertical axis and the time on the horizontal axis. For example, in the examples shown in FIGS. 12 through 15, as the color is closer to the direction of red (the direction toward the topmost color of the legend) and is darker (darker region), there are more frequency components, and as the color is closer to the direction of blue (the direction toward the bottommost color of the legend) and is darker (darker region), there are less frequency components.

The time-frequency analyzer 213 divides a sound waveform for 20 seconds, for example, by a predetermined unit of seconds (for example, 0.5 seconds), and performs Fourier transform on each of 0.5-second zones, thereby extracting a spectrogram. The time-frequency analyzer 213 supplies the spectrogram extracted from the sound waveform to the spectrogram determining section 33 as waveform feature information.

On the basis of such a spectrogram, the spectrogram determining section 33 is able to analyze how frequency components in each frequency range change over time. That is, the spectrogram determining section 33 is able to determine whether there is a periodicity (or the strength or the weakness of a periodicity) in each frequency range.

FIG. 12 is a diagram illustrating a spectrogram extracted as a result of the time-frequency analyzer 213 performing a short-time frequency analysis on breath sounds of a healthy person.

FIG. 13 is a diagram illustrating a spectrogram extracted as a result of the time-frequency analyzer 213 performing a short-time frequency analysis on decreased breath sounds.

FIG. 14 is a diagram illustrating a spectrogram extracted as a result of the time-frequency analyzer 213 performing a short-time frequency analysis on continuous adventitious sounds.

FIG. 15 is a diagram illustrating a spectrogram extracted as a result of the time-frequency analyzer 213 performing a short-time frequency analysis on discontinuous adventitious sounds.

The spectrogram determining section 33 analyzes the spectrogram shown in FIG. 12 and identifies that a strong periodicity is also observed in a range of 400 Hz or higher. That is, the spectrogram determining section 33 detects that a timing at which at least a certain number of signal components are generated (a relatively dark color portion) is observed at intervals of about three seconds in a range of 400 Hz or higher. As a result, the spectrogram determining section 33 is able to determine that a periodicity is also observed in a range of 400 Hz or higher in the spectrogram shown in FIG. 12.

Concerning the spectrogram shown in FIG. 13, the spectrogram determining section 33 determines that (a periodicity is not observed in a range of 400 Hz) a periodicity starts to be observed (intensified) in a range from 200 Hz to lower than 300 Hz. Unlike the spectrogram of normal breath sounds shown in FIG. 12, signal components in a high frequency range are not sufficiently observed in the spectrogram shown in FIG. 13 in which there is a sign of abnormal decreased breath sounds. A sign of abnormal decreased breath sounds is frequently observed in a case in which pleural effusion is stored between lungs and a thoracic cavity. The reason for this is as follows. If pleural effusion exists in a path from lungs in which normal breath sounds are generated until a stethoscope, this pleural effusion serves as a so-called low-pass filter and cuts high frequency components.

Concerning the spectrograms shown in FIGS. 14 and 15, the periodicity is weak or is not observed in any of frequency ranges. Accordingly, the spectrogram determining section 33 may determine in terms of the periodicity that the periodicity is weak as features of the sound waveform having such a spectrogram. However, the periodicity determining section 31 is able to determine the strength or the weakness of the periodicity from an autocorrelation function. Accordingly, if the waveform feature determining unit 30 includes the periodicity determining section 31, the spectrogram determining section 33 does not necessarily determine the strength or the weakness of the periodicity.

In this embodiment, the spectrogram determining section 33 reads waveform feature determination criteria stored in the storage unit 13 and applies them to the above-described spectrogram. Then, the spectrogram determining section 33 determines whether or not the spectrogram matches the waveform feature determination criteria. With this operation, the spectrogram determining section 33 is able to specify features of the sound waveform having this spectrogram in terms of the time-frequency components.

FIG. 16 illustrates examples of waveform feature determination criteria referred to by the spectrogram determining section 33 and examples of waveform feature determination results output from the spectrogram determining section 33.

In this embodiment, the spectrogram determining section 33 executes determination item 3-A or determination item 3-B in accordance with the waveform feature determination criteria shown in FIG. 16 and outputs waveform feature determination results. The spectrogram determining section 33 outputs a binary value, that is, true or false, concerning each of the determination items, as waveform feature determination results.

However, the content shown in FIG. 16 is only an example for explaining the functions of the spectrogram determining section 33, and it is not intended to restrict the configuration of the spectrogram determining section 33. Thresholds (values between “**_” and “_**”) defined in the waveform feature determination criteria shown in FIG. 16 may be changed and set as desired by a user (such as the operator U) of the information analyzing apparatus 100. Instead of using binary values, that is, true or false, the spectrogram determining section 33 may output waveform feature determination results with more details than binary values.

### (Determination Item 3-A: Determining Whether or not Periodicity is Observed in High Frequency Range)

The spectrogram determining section 33 executes determination item 3-A shown in FIG. 16. In determination item 3-A, the spectrogram determining section 33 determines whether or not a periodicity of at least a certain number of frequency components (darker portion) is observed at a frequency of 400 Hz (or higher) of the spectrogram. If a periodicity is observed in a range of 400 Hz or higher, the spectrogram determining section 33 determines determination item 3-A to be true and outputs “true” to the sound-type determining unit 40 as waveform feature determination results. If a periodicity is not observed in a range of 400 Hz or higher, the spectrogram determining section 33 determines determination item 3-A to be false and outputs “false” to the sound-type determining unit 40 as waveform feature determination results.

If a strong periodicity is observed in a range of 400 Hz or higher (if the waveform feature determination results indicate true), as shown in FIG. 12, the sound-type determining unit 40 is able to determine that the body sound information is substantially normal on the basis of the waveform feature determination results. In contrast, if the periodicity is weak or is not observed in a range of 400 Hz or higher (if the waveform feature determination results indicate false), as shown in FIGS. 13 through 15, the sound-type determining unit 40 is able to determine that there is a possibility of the occurrence of an abnormality (in particular, decreased breath sounds or adventitious sounds).

### (Determination Item 3-B: Determining Whether or not Periodicity Observed in Low Frequency Range is Weakened in High Frequency Range)

The spectrogram determining section 33 executes determination item 3-B shown in FIG. 16. In determination item 3-B, the spectrogram determining section 33 scans a spectrogram from a high frequency range (the scanning start point may be about 500 to 400 Hz) to a low frequency range, and specifies a frequency at which a periodicity stops to be observed (or starts to be weakened). Then, if the frequency at which the periodicity can be observed is lower than 400 Hz, the spectrogram determining section 33 determines determination item 3-B to be true, and if the frequency at which the periodicity can be observed is 400 Hz or higher, the spectrogram determining section 33 determines determination item 3-B to be false.

If the waveform feature determination results indicate false, that is, if a strong periodicity is observed in a range of 400 Hz or higher, it means that there is a periodicity in a high frequency range. Accordingly, if determination results of determination item 3-B indicating “false” are output, the sound-type determining unit 40 is able to determine that the possibility that the body sound waveform indicates decreased breath sounds is low. In contrast, if the waveform feature determination results indicate true, that is, if the frequency at which a strong periodicity can be observed is lower than 400 Hz, it means that a strong periodicity observed in a low frequency range is weakened (or not observed) in a high frequency range. Accordingly, if determination results of determination item 3-B indicating “true” are output, the sound-type determining unit 40 is able to determine that the possibility that the body sound waveform indicates decreased breath sounds is high.

If the spectrogram determining section 33 scans a spectrogram from a low frequency range (0 Hz) to a high frequency range, it may specify a frequency at which the periodicity has been weakened and disappeared. Then, if the frequency at which the periodicity has been weakened and disappeared is lower than 400 Hz, the spectrogram determining section 33 determines determination item 3-B to be true, and if the frequency at which the periodicity has been weakened and disappeared is 400 Hz or higher, the spectrogram determining section 33 determines determination item 3-B to be false.

If the frequency at which the periodicity has been weakened and disappeared is lower than 400 Hz (if determination item 3-B is true), as shown in FIG. 13, the sound-type determining unit 40 is able to determine that even though a strong periodicity is observed, the possibility that decreased breath sounds have been generated is high. In contrast, if the frequency at which the periodicity has been weakened and disappeared is 400 Hz or higher (for example, 900 Hz), that is, if determination item 3-B is false, as shown in FIG. 12, the sound-type determining unit 40 is able to determine that the possibility that decreased breath sounds have been generated is low and breath sounds are normal.

In the above-described example, the time-frequency analyzer 213 performs Fourier transform with a fixed temporal resolution, that is, the time-frequency analyzer 213 performs Fourier transform at fixed time intervals (for example, 0.5 seconds). However, the time-frequency analyzer 213 is not restricted to this configuration. The time-frequency analyzer 213 may perform wavelet transform so as to find a time-frequency component distribution. In wavelet transform, the temporal resolution may be changed for a low frequency and for a high frequency, thereby making it possible to obtain a more detailed time-frequency component distribution.

### (Feature Determining Function Based on Envelope)

FIG. 17 is a diagram illustrating a specific example of an envelope of a body sound waveform output from the envelope detector 214. The body sound waveform shown in FIG. 17 is obtained by enlarging part of the sound waveform of the body sound information shown in FIG. 9.

The envelope detector 214 of the body sound processor 21 detects and outputs an envelope of a sound waveform included in body sound information obtained by the body sound obtaining unit 20.

The envelope determining section 34 of the waveform feature determining unit 30 analyzes the envelope of the sound waveform output from the envelope detector 214 and applies waveform feature determination criteria to the envelope, thereby determining features of the sound waveform on the basis of the envelope.

If there are continuous adventitious sounds for 200 ms or longer in body sound information, it can be determined that the possibility of a symptom of asthma is high. As stated above when discussing a mechanism in which continuous adventitious sounds are generated, diseases related to continuous adventitious sounds include, not only asthma, but also obstructive lung disease (such as pulmonary emphysema and chronic obstructive pulmonary disease), and tracheal stenosis and bronchial stenosis. For simple representation, however, it is to be understood that a description will be given by taking asthma by way of example.

The generation of continuous adventitious sounds may originate from the fact that turbulence is continuously generated when the air flow passes through a respiratory tract in which secretion is stored due to asthma.

If part of the body sound waveform during a period from 6.6 to 7 seconds is enlarged and observed, as shown in FIG. 17, it is seen that the body sound information can be collected as a high frequency signal, as in AM modulation or FM modulation in a communication technology. In this case, in order to determine whether continuous adventitious sounds are generated for 200 ms or longer, a technique called envelope detection is desirably employed. Envelope detection performed by the envelope detector 214 is a technique used for demodulating AM-modulated signals and for extracting an envelope of a high frequency signal. The envelope detector 214 detects an envelope from a body sound waveform, which is a high frequency signal, and outputs the detected envelope to the envelope determining section 34.

The envelope determining section 34 is able to analyze the waveform of the envelope detected by the envelope detector 214 and to specify features of the sound waveform (for example, the length of adventitious sounds) as a feature quantity on the basis of the envelope.

FIG. 18 illustrates examples of waveform feature determination criteria referred to by the envelope determining section 34 and examples of waveform feature determination results output from the envelope determining section 34.

Part (a) of FIG. 19 is a diagram illustrating a specific example of an envelope having a high continuity, and part (b) of FIG. 19 is a diagram illustrating a specific example of an envelope having a low continuity.

In this embodiment, the envelope determining section 34 executes determination item 4 in accordance with the waveform feature determination criteria shown in FIG. 18 and outputs waveform feature determination results. The envelope determining section 34 outputs a binary value, that is, true or false, concerning the above-described determination item, as waveform feature determination results.

However, the content shown in FIG. 18 is only an example for explaining the functions of the envelope determining section 34, and it is not intended to restrict the configuration of the envelope determining section 34. Thresholds (values between “**_” and “_**”) defined in the waveform feature determination criteria shown in FIG. 18 may be changed and set as desired by a user (such as the operator U) of the information analyzing apparatus 100. Instead of using binary values, that is, true or false, the envelope determining section 34 may output waveform feature determination results with more details than binary values.

### (Determination Item 4: Determining Whether or not the Continuity of Adventitious Sounds is Observed)

The envelope determining section 34 executes determination item 4 shown in FIG. 18. In determination item 4, the envelope determining section 34 determines whether the continuity of sounds is observed in an envelope of a sound waveform.

The envelope determining section 34 first performs determination item 4-1. In determination item 4-1, the envelope determining section 34 determines whether or not a time for which the amplitude of an envelope of a sound waveform exceeds the amplitude average value continues for 200 ms or longer.

For example, the envelope shown in part (a) of FIG. 19 will be discussed by way of example. The amplitude average value of the envelope is indicated by the long dashed dotted line Avr1. In this case, the envelope determining section 34 specifies a zone in which the amplitude exceeds the amplitude average value Avr1 as Z1. The length of the zone Z1 is 200 ms or longer. Accordingly, when executing determination item 4-1 concerning the envelope shown in part (a) of FIG. 19, the envelope determining section 34 outputs “true (time continues for 200 ms or longer)” as waveform feature determination results.

Then, the envelope shown in part (b) of FIG. 19 will be discussed by way of example. The amplitude average value of the envelope is indicated by the long dashed dotted line Avr2. In this case, the envelope determining section 34 specifies zones in which the amplitude exceeds the amplitude average value Avr2 as Z2, Z3, and Z4. None of the lengths of the zones Z2, Z3, and Z4 are 200 ms or longer. Accordingly, when executing determination item 4 concerning the envelope shown in part (b) of FIG. 19, the envelope determining section 34 outputs “false (time does not continue for 200 ms or longer)” as waveform feature determination results.

Then, the envelope determining section 34 executes determination item 4-2. In determination item 4-2, the envelope determining section 34 determines whether or not a total time for which the amplitude of a sound waveform during one period (about two to five seconds) of breath sounds exceeds the amplitude average value in the envelope of this sound waveform is 200 ms or longer. For example, the envelope determining section 34 adds the times of zones for which the amplitude exceeds the amplitude average value Avr2 in the envelope during one period of breath sounds. In the example shown in part (b) of FIG. 19, the envelope determining section 34 adds the times of zones Z2, Z3, Z4, and so on. If the total time is 200 ms or longer, the envelope determining section 34 returns “true” in accordance with determination item 4-2, in a manner different from determination item 4-1.

Then, the envelope determining section 34 outputs “true (the total time is 200 ms or longer)” or “false (the total time is shorter than 200 ms)” as waveform feature determination results of determination item 4-2.

Finally, the envelope determining section 34 integrates the results of determination item 4-1 and determination item 4-2 and outputs the waveform feature determination results of determination item 4. For example, if at least one of determination item 4-1 and determination item 4-2 is true, the envelope determining section 34 may determine determination item 4 to be true (the continuity of sounds is observed) and may output “true” as the waveform feature determination results of determination item 4 based on the envelope. If both of determination item 4-1 and determination item 4-2 are false, the envelope determining section 34 may determine determination item 4 to be false (the continuity of sounds is not observed) and may output “false” as the waveform feature determination results of determination item 4 based on the envelope.

If determination item 4 is true, the sound-type determining unit 40 is able to determine that the continuity of adventitious sounds is high, that is, there may be a possibility that continuous adventitious sounds have been generated, on the basis of the waveform feature determination results. On the other hand, if determination item 4 is false, the sound-type determining unit 40 is able to determine that the continuity of adventitious sounds is low, that is, there is a possibility that continuous adventitious sounds have not been generated, on the basis of the waveform feature determination results. In this manner, as a result of the envelope determining section 34 integrating determination item 4-1 and determination item 4-2 and outputting waveform feature determination results, the sound-type determining unit 40 is able to more precisely determine the sound type in terms of the continuity of sounds.

### (Feature Determining Function Based on Impulse Noise)

FIG. 20 is a diagram illustrating a specific example of impulse noise detection results, in which impulse noise is specified in a waveform of body sounds, output from the impulse noise detector 215.

The impulse noise detector 215 of the body sound processor 21 detects impulse noise included in a sound waveform of body sound information obtained by the body sound obtaining unit 20. The impulse noise detector 215 outputs impulse noise detection results to the impulse noise determining section 35.

The impulse noise determining section 35 of the waveform feature determining unit 30 applies waveform feature determination criteria to the impulse noise detection results supplied from the impulse noise detector 215 so as to determine features of the sound waveform on the basis of the number of noise components (feature quantity).

The impulse noise detection results may be a data structure, as shown in FIG. 20, in which impulse noise is emphasized in a body sound waveform and is thus easy to recognize by the impulse noise determining section 35. Alternatively, the impulse noise detection results may be information simply indicating how many impulse noise components have been detected in a body sound waveform.

Impulse noise is instantaneously generated burst noise. The burst noise is generated due to the fact that a liquid film blocking a respiratory tract bursts when the air flow passes through the respiratory tract. Accordingly, a patient emitting breath sounds in which many impulse noise components are detected may suffer from a disease showing a symptom such as a respiratory tract being blocked by a liquid film (for example, pneumonia or sputum retention).

FIG. 21 illustrates examples of waveform feature determination criteria referred to by the impulse noise determining section 35 and examples of waveform feature determination results output from the impulse noise determining section 35.

In this embodiment, the impulse noise determining section 35 executes determination item 5 in accordance with the waveform feature determination criteria shown in FIG. 21 and outputs waveform feature determination results. The impulse noise determining section 35 outputs a binary value, that is, true or false, concerning the above-described determination item, as waveform feature determination results.

However, the content shown in FIG. 21 is only an example for explaining the functions of the impulse noise determining section 35, and it is not intended to restrict the configuration of the impulse noise determining section 35. Thresholds (values between “**_” and “_**”) defined in the waveform feature determination criteria shown in FIG. 21 may be changed and set as desired by a user (such as the operator U) of the information analyzing apparatus 100. Instead of using binary values, that is, true or false, the impulse noise determining section 35 may output waveform feature determination results with more details than binary values.

### (Determination Item 5: Determining Whether or not the Discontinuity of Adventitious Sounds is Observed)

The impulse noise determining section 35 executes determination item 5 shown in FIG. 21. In determination item 5, the impulse noise determining section 35 determines whether or not the number of impulse noise components included in a sound waveform per period is ten or more.

The impulse noise determining section 35 may calculate the number of impulse noise components for five seconds as the number of impulse noise components per period, on the basis of the number of impulse noise components for the total time (seconds) of a body sound waveform. With this arrangement, even for a sound waveform exhibiting a weak periodicity, the number of impulse noise components per period can be specified. For example, if a strong periodicity is not observed in a body sound waveform for 20 seconds, the impulse noise determining section 35 obtains total impulse noise components included in the body sound waveform from the impulse noise detector 215. For example, if the total number of impulse noise components is 32, the impulse noise determining section 35 may specify the number of impulse noise components per period to be eight (32÷(20 seconds÷5 seconds)=8).

If the number of impulse noise components per period is ten or more, the impulse noise determining section 35 outputs “true” to the sound-type determining unit 40 as waveform feature determination results. If the number of impulse noise components per period is less than ten, the impulse noise determining section 35 outputs “false” to the sound-type determining unit 40 as waveform feature determination results.

If determination item 5 is true, the sound-type determining unit 40 is able to determine that the discontinuity of adventitious sounds is high, on the basis of the waveform feature determination results. On the other hand, if determination item 5 is false, the sound-type determining unit 40 is able to determine that the discontinuity of adventitious sounds is low, on the basis of the waveform feature determination results.

The individual elements of the sound-type determining unit 40 will be discussed below in detail.

### (Normal-Breath-Sound Determining Function)

FIG. 22 is a diagram illustrating a specific example of sound-type determination results which are output from the normal-breath-sound determining section 41 of the sound-type determining unit 40 by using, as input, waveform feature determination results output from the waveform feature determining unit 30.

The normal-breath-sound determining section 41 of the sound-type determining unit 40 determines whether or not body sounds included in body sound information obtained by the body sound obtaining unit 20 are classified as normal breath sounds. More specifically, in this embodiment, the normal-breath-sound determining section 41 outputs binary information indicating “true: there is a possibility that body sounds are normal breath sounds” or “false: there is a possibility that body sounds are not normal breath sounds” as sound-type determination results. The output sound-type determination results are supplied to the result output unit 23.

As shown in FIG. 22, in order to make a determination of “true” or “false”, the normal-breath-sound determining section 41 obtains waveform feature determination results concerning determination item 1, determination item 2-A, and determination item 3-A from the waveform feature determining unit 30.

More specifically, the normal-breath-sound determining section 41 obtains waveform feature determination results concerning determination item 1 indicating the strength or the weakness of a periodicity from the periodicity determining section 31. The normal-breath-sound determining section 41 obtains waveform feature determination results concerning determination item 2-A indicating the normality of a frequency component distribution from the spectrum determining section 32. The normal-breath-sound determining section 41 also obtains waveform feature determination results concerning determination item 3-A indicating the presence or the absence (or the strength or the weakness) of a periodicity in a high frequency range from the spectrogram determining section 33.

As a result of obtaining binary information, that is, true or false, concerning the above-described three determination items, eight patterns (a) through (h) of combinations of “true” and “false” can be considered, as shown in FIG. 22. The normal-breath-sound determining section 41 makes a determination of “true” or “false” concerning normal breath sounds for each of the eight patterns.

In this embodiment, as shown in FIG. 22, only in the case of pattern (a) in which all the determination items are true, the normal-breath-sound determining section 41 makes a determination of “true: there is a possibility that body sounds are normal breath sounds”. If there is even one “false” among the three determination items, the normal-breath-sound determining section 41 makes a determination of “false: there is a possibility that body sounds are not normal breath sounds”.

As discussed above, body sounds (respiratory system sounds) for which determination item 1 is true are considered to have a strong periodicity. Body sounds for which determination item 2-A is true are considered to have a substantially normal frequency component distribution. Body sounds for which determination item 3-A is true are considered to have a periodicity (or a strong periodicity) in a high frequency range. Accordingly, in this embodiment, the normal-breath-sound determining section 41 concludes that body sounds for which all of these determination items are true are “true: there is a possibility that body sounds are normal breath sounds”. In contrast, body sounds for which determination item 1 is false are considered to have a weak periodicity. Body sounds for which determination item 2-A is false are considered to have an abnormal frequency component distribution. Body sounds for which determination item 3-A is false are considered to have no periodicity (or to have a weak periodicity) in a high frequency range. Accordingly, in this embodiment, body sounds for which even one of these determination results is false may have a certain abnormality, and thus, the normal-breath-sound determining section 41 concludes that such body sounds are “false: there is a possibility that body sounds are not normal breath sounds”.

The sound-type determination results output from the normal-breath-sound determining section 41 are displayed in the display unit 12 by the result output unit 23. For example, as shown in FIG. 29, if the normal-breath-sound determining section 41 outputs “true”, the result output unit 23 may display a message, such as “there is a possibility that breath sounds are normal”, in the display unit 12. In contrast, if the normal-breath-sound determining section 41 outputs “false”, the result output unit 23 may display a message, such as “there is a possibility that breath sounds are not normal”, in the display unit 12.

With this operation, analysis results of body sound information collected by a stethoscope can be provided to a user such that they are easy to understand.

### (Decreased-Breath-Sound Determining Function)

FIG. 23 is a diagram illustrating a specific example of sound-type determination results which are output from the decreased-breath-sound determining section 42 of the sound-type determining unit 40 by using, as input, waveform feature determination results output from the waveform feature determining unit 30.

The decreased-breath-sound determining section 42 of the sound-type determining unit 40 determines whether or not body sounds included in body sound information obtained by the body sound obtaining unit 20 are classified as decreased breath sounds. More specifically, in this embodiment, the decreased-breath-sound determining section 42 outputs binary information indicating “true: there is a possibility that body sounds are decreased breath sounds” or “false: there is a possibility that body sounds are not decreased breath sounds” as sound-type determination results. The output sound-type determination results are supplied to the result output unit 23.

As shown in FIG. 23, in order to make a determination of “true” or “false”, the decreased-breath-sound determining section 42 obtains waveform feature determination results concerning determination item 1, determination item 2-A, and determination item 3-B from the waveform feature determining unit 30.

More specifically, the decreased-breath-sound determining section 42 obtains waveform feature determination results concerning determination item 1 indicating the strength or the weakness of a periodicity from the periodicity determining section 31. The decreased-breath-sound determining section 42 obtains waveform feature determination results concerning determination item 2-A indicating the normality of a frequency component distribution from the spectrum determining section 32. The decreased-breath-sound determining section 42 also obtains waveform feature determination results concerning determination item 3-B indicating whether or not a strong periodicity observed in a low frequency range is weakened in a high frequency range from the spectrogram determining section 33.

As a result of obtaining binary information, that is, true or false, concerning the above-described three determination items, eight patterns (a) through (h) of combinations of “true” and “false” can be considered, as shown in FIG. 23. The decreased-breath-sound determining section 42 makes a determination of “true” or “false” concerning decreased breath sounds for each of the eight patterns.

In this embodiment, as shown in FIG. 23, only in the case of pattern (a) in which all the determination items are true, the decreased-breath-sound determining section 42 makes a determination of “true: there is a possibility that body sounds are decreased breath sounds”. If there is even one “false” among the three determination items, the decreased-breath-sound determining section 42 makes a determination of “false: there is a possibility that body sounds are not decreased breath sounds”. In this case, “body sounds are not decreased breath sounds” suggests that body sounds are normal or may have an abnormality other than decreased breath sounds.

As discussed above, body sounds (respiratory system sounds) for which determination item 1 is true are considered to have a strong periodicity. Body sounds for which determination item 2-A is true are considered to have a substantially normal frequency component distribution. Body sounds for which determination item 3-B is true are considered that a periodicity observed in a low frequency range is no longer observed (or is weakened) in a high frequency range. This feature observed in determination item 3-B is a typical symptom of decreased breath sounds. Accordingly, in this embodiment, the decreased-breath-sound determining section 42 concludes that that body sounds for which all of these determination items are true are “true: there is a possibility that body sounds are decreased breath sounds”.

In contrast, body sounds for which determination item 1 is false are considered to have a weak periodicity. Body sounds for which determination item 2-A is false are considered to have an abnormal frequency component distribution. Body sounds for which determination item 3-B is false are considered to have a periodicity (or a strong periodicity) even in a high frequency range. Accordingly, in this embodiment, body sounds for which even one of these determination results is false may have a characteristic different from a symptom of decreased breath sounds, and thus, the decreased-breath-sound determining section 42 concludes that such body sounds are “false: there is a possibility that body sounds are not decreased breath sounds”. The reason why there is a characteristic different from a symptom of decreased breath sounds may be that breath sounds are normal or have an abnormality other than decreased breath sounds.

The sound-type determination results output from the decreased-breath-sound determining section 42 are displayed in the display unit 12 by the result output unit 23. For example, as shown in FIG. 29, if the decreased-breath-sound determining section 42 outputs “true”, the result output unit 23 may display a message, such as “there is a possibility that body sounds are decreased breath sounds”, in the display unit 12. In contrast, if the decreased-breath-sound determining section 42 outputs “false”, the result output unit 23 may display a message, such as “there is a possibility that body sounds are not decreased breath sounds”, in the display unit 12.

With this operation, analysis results of body sound information collected by a stethoscope can be provided to a user such that they are easy to understand.

### (Continuous-Adventitious-Sound Determining Function)

FIG. 24 is a diagram illustrating a specific example of sound-type determination results which are output from the continuous-adventitious-sound determining section 43 of the sound-type determining unit 40 by using, as input, waveform feature determination results output from the waveform feature determining unit 30.

The continuous-adventitious-sound determining section 43 of the sound-type determining unit 40 determines whether or not body sounds included in body sound information obtained by the body sound obtaining unit 20 are classified as continuous adventitious sounds. More specifically, in this embodiment, the continuous-adventitious-sound determining section 43 outputs binary information indicating “true: there is a possibility that body sounds are continuous adventitious sounds” or “false: there is a possibility that body sounds are not continuous adventitious sounds” as sound-type determination results. The output sound-type determination results are supplied to the result output unit 23.

As shown in FIG. 24, in order to make a determination of “true” or “false”, the continuous-adventitious-sound determining section 43 obtains waveform feature determination results concerning determination item 1′, determination item 2-B, and determination item 4 from the waveform feature determining unit 30.

More specifically, the continuous-adventitious-sound determining section 43 obtains waveform feature determination results concerning determination item 1′ indicating whether or not the periodicity is weak from the periodicity determining section 31. The continuous-adventitious-sound determining section 43 obtains waveform feature determination results concerning determination item 2-B indicating the abnormality of a frequency component distribution from the spectrum determining section 32. The continuous-adventitious-sound determining section 43 also obtains waveform feature determination results concerning determination item 4 indicating whether or not the continuity of adventitious sounds is observed from the envelope determining section 34.

As a result of obtaining binary information, that is, true or false, concerning the above-described three determination items, eight patterns (a) through (h) of combinations of “true” and “false” can be considered, as shown in FIG. 24. The continuous-adventitious-sound determining section 43 makes a determination of “true” or “false” concerning continuous adventitious sounds for each of the eight patterns.

In this embodiment, as shown in FIG. 24, only in the case of pattern (a) in which all the determination items are true, the continuous-adventitious-sound determining section 43 makes a determination of “true: there is a possibility that body sounds are continuous adventitious sounds”. If there is even one “false” among the three determination items, the continuous-adventitious-sound determining section 43 makes a determination of “false: there is a possibility that body sounds are not continuous adventitious sounds”. In this case, “body sounds are not continuous adventitious sounds” suggests that breath sounds may be normal or may have an abnormality other than continuous adventitious sounds.

As discussed above, body sounds (respiratory system sounds) for which determination item 1′ is true are considered to have a weak periodicity. Body sounds for which determination item 2-B is true are considered to have a substantially abnormal frequency component distribution. Body sounds for which determination item 4 is true are considered that the continuity of adventitious sounds is observed. This feature concerning determination item 4 is a typical symptom of continuous adventitious sounds. Accordingly, in this embodiment, the continuous-adventitious-sound determining section 43 concludes that body sounds for which all of these determination items are true are “true: there is a possibility that body sounds are continuous adventitious sounds”.

In contrast, body sounds for which determination item 1′ is false are considered to have a strong periodicity. Body sounds for which determination item 2-B is false are considered not to have an abnormal frequency component distribution. Body sounds for which determination item 4 is false are considered that the continuity of adventitious sounds is not observed. Accordingly, in this embodiment, body sounds for which even one of these determination results is false may have a characteristic different from a symptom of continuous adventitious sounds, and thus, the continuous-adventitious-sound determining section 43 concludes that such body sounds are “false: there is a possibility that body sounds are not continuous adventitious sounds”. The reason why there is a characteristic different from a symptom of continuous adventitious sounds may be that breath sounds are normal or have an abnormality other than continuous adventitious sounds.

The sound-type determination results output from the continuous-adventitious-sound determining section 43 are displayed in the display unit 12 by the result output unit 23. For example, as shown in FIG. 29, if the continuous-adventitious-sound determining section 43 outputs “true”, the result output unit 23 may display a message, such as “there is a possibility that body sounds are continuous adventitious sounds”, in the display unit 12. In contrast, if the continuous-adventitious-sound determining section 43 outputs “false”, the result output unit 23 may display a message, such as “there is a possibility that body sounds are not continuous adventitious sounds”, in the display unit 12.

With this operation, analysis results of body sound information collected by a stethoscope can be provided to a user such that they are easy to understand.

### (Discontinuous-Adventitious-Sound Determining Function)

FIG. 25 is a diagram illustrating a specific example of sound-type determination results which are output from the discontinuous-adventitious-sound determining section 44 of the sound-type determining unit 40 by using, as input, waveform feature determination results output from the waveform feature determining unit 30.

The discontinuous-adventitious-sound determining section 44 of the sound-type determining unit 40 determines whether or not body sounds included in body sound information obtained by the body sound obtaining unit 20 are classified as discontinuous adventitious sounds. More specifically, in this embodiment, the discontinuous-adventitious-sound determining section 44 outputs binary information indicating “true: there is a possibility that body sounds are discontinuous adventitious sounds” or “false: there is a possibility that body sounds are not discontinuous adventitious sounds” as sound-type determination results. The output sound-type determination results are supplied to the result output unit 23.

As shown in FIG. 25, in order to make a determination of “true” or “false”, the discontinuous-adventitious-sound determining section 44 obtains waveform feature determination results concerning determination item 1′, determination item 2-B, and determination item 5 from the waveform feature determining unit 30.

More specifically, the discontinuous-adventitious-sound determining section 44 obtains waveform feature determination results concerning determination item 1′ indicating whether or not the periodicity is weak from the periodicity determining section 31. The discontinuous-adventitious-sound determining section 44 obtains waveform feature determination results concerning determination item 2-B indicating the abnormality of a frequency component distribution from the spectrum determining section 32. The discontinuous-adventitious-sound determining section 44 also obtains waveform feature determination results concerning determination item 5 indicating whether or not the discontinuity of adventitious sounds is observed from the impulse noise determining section 35.

As a result of obtaining binary information, that is, true or false, concerning the above-described three determination items, eight patterns (a) through (h) of combinations of “true” and “false” can be considered, as shown in FIG. 25. The discontinuous-adventitious-sound determining section 44 makes a determination of “true” or “false” concerning discontinuous adventitious sounds for each of the eight patterns.

In this embodiment, as shown in FIG. 25, only in the case of pattern (a) in which all the determination items are true, the discontinuous-adventitious-sound determining section 44 makes a determination of “true: there is a possibility that body sounds are discontinuous adventitious sounds”. If there is even one “false” among the three determination items, the discontinuous-adventitious-sound determining section 44 makes a determination of “false: there is a possibility that body sounds are not discontinuous adventitious sounds”. In this case, “body sounds are not discontinuous adventitious sounds” suggests that breath sounds may be normal or may have an abnormality other than discontinuous adventitious sounds.

As discussed above, body sounds (respiratory system sounds) for which determination item 1′ is true are considered to have a weak periodicity. Body sounds for which determination item 2-B is true are considered to have a substantially abnormal frequency component distribution. Body sounds for which determination item 5 is true are considered that many discontinuous adventitious sounds (impulse noise components) are observed. This feature concerning determination item 5 is a typical symptom of discontinuous adventitious sounds. Accordingly, in this embodiment, the discontinuous-adventitious-sound determining section 44 concludes that body sounds for which all of these determination items are true are “true: there is a possibility that body sounds are discontinuous adventitious sounds”.

In contrast, body sounds for which determination item 1′ is false are considered to have a strong periodicity. Body sounds for which determination item 2-B is false are considered not to have an abnormal frequency component distribution. Body sounds for which determination item 5 is false are considered that not many impulse noise components are observed. Accordingly, in this embodiment, body sounds for which even one of these determination results is false may have a characteristic different from a symptom of discontinuous adventitious sounds, and thus, the discontinuous-adventitious-sound determining section 44 concludes that such body sounds are “false: there is a possibility that body sounds are not discontinuous adventitious sounds”. The reason why there is a characteristic different from a symptom of discontinuous adventitious sounds may be that breath sounds are normal or have an abnormality other than discontinuous adventitious sounds.

The sound-type determination results output from the discontinuous-adventitious-sound determining section 44 are displayed in the display unit 12 by the result output unit 23. For example, as shown in FIG. 29, if the discontinuous-adventitious-sound determining section 44 outputs “true”, the result output unit 23 may display a message, such as “there is a possibility that body sounds are discontinuous adventitious sounds”, in the display unit 12. In contrast, if the discontinuous-adventitious-sound determining section 44 outputs “false”, the result output unit 23 may display a message, such as “there is a possibility that body sounds are not discontinuous adventitious sounds”, in the display unit 12.

With this operation, analysis results of body sound information collected by a stethoscope can be provided to a user such that they are easy to understand.

In this embodiment, as shown in FIG. 29, an example in which the result output unit 23 displays all sound-type determination results obtained by the individual determining sections of the sound-type determining unit 40 has been discussed. However, the information analyzing apparatus 100 of the present invention is not restricted to this configuration. For example, if breath sounds are classified as normal breath sounds by the normal body sound determining section 41 and if determination results concerning abnormal sounds obtained by the other determining sections of the sound-type determining unit 40 are all false (breath sounds are not abnormal), the result output unit 23 may display analysis results by omitting sound-type determination results obtained by the other determining sections of the sound-type determining unit 40.

In contrast, the following case may be assumed. A plurality of abnormal sound determining sections (the decreased-breath-sound determining section 42, the continuous-adventitious-sound determining section 43, and the discontinuous-adventitious-sound determining section 44) other than the normal-breath-sound determining section 41 determine that breath sounds are abnormal. In this case, regardless of whether or not the normal-breath-sound determining section 41 has determined that breath sounds are normal, the result output unit 23 may separately display a message used for multiple abnormalities, such as “there is a possibility that multiple diseases may be concurrently occurring”, in addition to messages concerning individual abnormal sounds, such as “there is a possibility that body sounds are xxx sounds”. For example, if breath sounds are decreased breath sounds and also continuous adventitious sounds, the result output unit 23 may display both of messages “there is a possibility that body sounds are decreased breath sounds” and “there is a possibility that body sounds are continuous adventitious sounds” at the same time, and may also display a message “there is a possibility that multiple diseases are concurrently occurring”.

### (Abnormality Appearance Frequency Determining Function)

As shown in FIG. 29, each determining section of the sound-type determining unit 40 may count the number of times (frequency) which a corresponding type of abnormality appears in all sound waveforms included in body sound information, and may output the counted number of times to the result output unit 23. For example, the continuous-adventitious-sound determining section 43 may analyze body sound waveforms for 40 seconds (equal to about ten breathing periods), and may count how many waveforms that match the determination pattern (a) shown in FIG. 24 have been detected. Then, the continuous-adventitious-sound determining section 43 may supply, together with sound-type determination results, information concerning the number of times continuous adventitious sounds have been detected to the result output unit 23.

The individual elements of the abnormality-level determining unit 50 will be discussed below in detail. The information analyzing apparatus 100 of the present invention does not necessarily include the abnormality-level determining unit 50. However, in case that the sound-type determining unit 40 classifies body sounds as an abnormal sound type, it is preferable that the abnormality-level determining unit 50 for determining the degree (level) of such an abnormality is provided.

### (Decreased-Sound-Level Determining Function)

The decreased-sound-level determining section 51 determines a decreased sound level of a waveform of body sounds which are determined to be “true: there is a possibility that body sounds are decreased breath sounds” by the decreased-breath-sound determining section 42.

FIG. 26 illustrates examples of decreased-sound-level determination criteria referred to by the decreased-sound-level determining section 51 and examples of decreased-sound-level determination results output from the decreased-sound-level determining section 51.

If the decreased-breath-sound determining section 42 determines that “there is a possibility that body sounds are decreased breath sounds”, the decreased-sound-level determining section 51 determines the level of decreased sounds. More specifically, the decreased-sound-level determining section 51 reads decreased-sound-level determination criteria stored in the storage unit 13 shown in FIG. 26. Then, the decreased-sound-level determining section 51 applies the read criteria to a spectrogram of body sounds output from the time-frequency analyzer 213. Then, the decreased-sound-level determining section 51 determines the decreased sound level of the body sounds, depending on which criterion the sound waveform matches. In this embodiment, the decreased-sound-level determining section 51 outputs decreased-sound-level determination results in three levels, such as “low”, “intermediate”, and “high” by way of example.

“Low” means that the degree of decreased sounds is comparatively light, “high” means that the degree of decreased sounds is comparatively heavy, and “intermediate” is a level between “low” and “high”. As more high-frequency components are cut with a wider range, the degree of decreased sounds is heavier.

The content shown in FIG. 26 is only an example for explaining the functions of the decreased-sound-level determining section 51, and it is not intended to restrict the configuration of the decreased-sound-level determining section 51. Thresholds (values between “**_” and “_**”) defined in the decreased-sound-level determination criteria shown in FIG. 26 may be changed and set as desired by a user (such as the operator U) of the information analyzing apparatus 100. Instead of three values, such as “low”, “intermediate”, and “high”, the decreased-sound-level determining section 51 may output decreased-sound-level determination results with more detailed multilevel values. Alternatively, the decreased-sound-level determining section 51 may simply output two values, such as “low (light)” and “high (heavy)”.

As shown in FIG. 26, the decreased-sound-level determining section 51 first specifies, from a spectrogram, the frequency at a boundary between a frequency range in which a periodicity (a strong periodicity) is observed and a frequency range in which a periodicity is not observed (a weak periodicity is observed). As in the spectrogram determining section 33, the decreased-sound-level determining section 51 may scan the spectrogram so as to detect this boundary. Alternatively, if the spectrogram determining section 33 has already specified the boundary, the decreased-sound-level determining section 51 may obtain the frequency value at this boundary from the spectrogram determining section 33. For example, in the example shown in FIG. 13, the decreased-sound-level determining section 51 determines that the frequency at the boundary is about 330 Hz.

Then, the decreased-sound-level determining section 51 reads the decreased-sound-level determination criteria shown in FIG. 26 and determines which criterion the spectrogram having the above-described boundary matches. In the examples shown in FIGS. 13 and 26, the decreased-sound-level determining section 51 determines that the boundary (the frequency at which a strong periodicity has disappeared (weakened)) is in a range from 300 Hz to 400 Hz.

Finally, the decreased-sound-level determining section 51 outputs the decreased sound level (low) corresponding to the determined results to the result output unit 23 as decreased-sound-level determination results.

The decreased-sound-level determination results output from the decreased-sound-level determining section 51 are displayed in the display unit 12 by the result output unit 23. For example, in a region of the display unit 12 shown in FIG. 29 in which level determination results are displayed, a message, such as “• decreased sound level: low” may be displayed.

With this operation, analysis results of body sound information collected by a stethoscope can be provided to a user such that they are easy to understand. That is, not only results indicating whether body sounds are normal or abnormal, but also, if body sounds are abnormal, the degree (level) of the abnormality can be provided to a user such that they are easy to understand.

### (Continuity-Level Determining Function)

The continuity-level determining section 52 determines the level of the continuity of a waveform of body sounds which are determined to be “true: there is a possibility that body sounds are continuous adventitious sounds” by the continuous-adventitious-sound determining section 43.

FIG. 27 illustrates examples of continuity-level determination criteria referred to by the continuity-level determining section 52 and examples of continuity-level determination results output from the continuity-level determining section 52.

If the continuous-adventitious-sound determining section 43 determines that “there is a possibility that body sounds are continuous adventitious sounds”, the continuity-level determining section 52 determines a continuity level. More specifically, the continuity-level determining section 52 reads the continuity-level determination criteria stored in the storage unit 13 shown in FIG. 27. Then, the continuity-level determining section 52 applies the read criteria to an envelope of the body sounds output from the envelope detector 214. Then, the continuity-level determining section 52 determines the level of the continuity of the body sounds, depending on which criterion the sound waveform matches. In this embodiment, the continuity-level determining section 52 outputs continuity-level determination results in three levels, such as “low”, “intermediate”, and “high” by way of example.

“Low” means that the degree of the continuity is comparatively light, “high” means that the degree of the continuity is comparatively heavy, and “intermediate” is a level between “low” and “high”. As a waveform having a greater amplitude value continues for a longer time in an envelope, the degree of the continuity is heavier.

The content shown in FIG. 27 is only an example for explaining the functions of the continuity-level determining section 52, and it is not intended to restrict the configuration of the continuity-level determining section 52. Thresholds (values between “**_” and “_**”) defined in the continuity-level determination criteria shown in FIG. 27 may be changed and set as desired by a user (such as the operator U) of the information analyzing apparatus 100. Instead of three values, such as “low”, “intermediate”, and “high”, the continuity-level determining section 52 may output continuity-level determination results with more detailed multilevel values. Alternatively, the continuity-level determining section 52 may simply output two values, such as “low (light)” and “high (heavy)”.

As shown in FIG. 27, the continuity-level determining section 52 first specifies, from a detected envelope, the length of a continuous zone (time) in which the amplitude exceeds the amplitude average value. As in the envelope determining section 34, the continuity-level determining section 52 may specify a zone Z in which the amplitude exceeds the amplitude average value in the envelope and may also specify the time length of the zone Z. Alternatively, if the envelope determining section 34 has already specified the time length of the zone Z, the continuity-level determining section 52 may obtain the time length from the envelope determining section 34. For example, in the example shown in part (a) of FIG. 19, the continuity-level determining section 52 specifies the time length of the zone Z1 to be 250 ms. If there are multiple zones in which the amplitude exceeds the amplitude average value Avr, such as in the example shown in part (b) of FIG. 19, the continuity-level determining section 52 may specify the average time length of the zones 2 through 4, or the longest time length among those of the zones 2 through 4.

Then, the continuity-level determining section 52 reads the continuity-level determination criteria shown in FIG. 27 and determines which criterion the specified time length matches. In the examples shown in part (a) of FIG. 19 and FIG. 27, since the specified time length is 250 ms, the continuity-level determining section 52 determines that the specified time length is from 200 ms to shorter than 600 ms.

Finally, the continuity-level determining section 52 outputs the continuity level (low) corresponding to the determined results to the result output unit 23 as continuity-level determination results.

The continuity-level determination results output from the continuity-level determining section 52 are displayed in the display unit 12 by the result output unit 23. For example, as shown in FIG. 29, in a region of the display unit 12 in which level determination results are displayed, a message, such as “• continuity level: low” may be displayed.

With this operation, analysis results of body sound information collected by a stethoscope can be provided to a user such that they are easy to understand. That is, not only results indicating whether body sounds are normal or abnormal, but also, if body sounds are abnormal, the degree (level) of the abnormality can be provided to a user such that it is easy to understand.

### (Discontinuity Level Determining Function)

The discontinuity-level determining section 53 determines the level of the discontinuity of a waveform of body sounds which are determined to be “true: there is a possibility that body sounds are discontinuous adventitious sounds” by the discontinuous-adventitious-sound determining section 44.

FIG. 28 illustrates examples of discontinuity-level determination criteria referred to by the discontinuity-level determining section 53 and examples of discontinuity-level determination results output from the discontinuity-level determining section 53.

If the discontinuous-adventitious-sound determining section 44 determines that “there is a possibility that body sounds are discontinuous adventitious sounds”, the discontinuity-level determining section 53 determines the level of the discontinuity. More specifically, the discontinuity-level determining section 53 reads the discontinuity-level determination criteria stored in the storage unit 13 shown in FIG. 28. Then, the discontinuity-level determining section 53 applies the read criteria to impulse noise detection results concerning the body sounds output from the impulse noise detector 215. Then, the discontinuity-level determining section 53 determines the level of the discontinuity of the body sounds, depending on which criterion the sound waveform matches. In this embodiment, the discontinuity-level determining section 53 outputs discontinuity-level determination results in three levels, such as “low”, “intermediate”, and “high” by way of example.

“Low” means that the degree of the discontinuity is comparatively light, “high” means that the degree of the discontinuity is comparatively heavy, and “intermediate” is a level between “low” and “high”. In impulse noise detection results, as more impulse noise components are detected, the degree of the discontinuity is heavier.

The content shown in FIG. 28 is only an example for explaining the functions of the discontinuity-level determining section 53, and it is not intended to restrict the configuration of the discontinuity-level determining section 53. Thresholds (values between “**_” and “_**”) defined in the discontinuity-level determination criteria shown in FIG. 28 may be changed and set as desired by a user (such as the operator U) of the information analyzing apparatus 100. Instead of three values, such as “low”, “intermediate”, and “high”, the discontinuity-level determining section 53 may output discontinuity-level determination results with more detailed multilevel values. Alternatively, the discontinuity-level determining section 53 may simply output two values, such as “low (light)” and “high (heavy)”.

The discontinuity-level determining section 53 first specifies how many impulse noise components have been detected per period in the impulse noise detection results. As in the impulse noise determining section 35, the discontinuity-level determining section 53 may specify the number of impulse noise components per period from the impulse noise detection results. Alternatively, if the impulse noise determining section 35 has already specified the number of impulse noise components per period, the discontinuity-level determining section 53 may obtain the number of impulse noise components from the impulse noise determining section 35.

For example, in the example shown in FIG. 20, five impulse noise components are contained during 0.5 seconds from 7.5 to 8 seconds, and in terms of one period (set to be about 5 seconds), 50 impulse noise components are detected. Thus, the discontinuity-level determining section 53 may specify the number of impulse noise components of the body sounds per period to be 50.

Then, the discontinuity-level determining section 53 reads the discontinuity-level determination criteria shown in FIG. 28 and determines which criterion the specified number of impulse noise components matches. In the examples shown in FIGS. 20 and 28, since the specified number of impulse noise components is 50, the discontinuity-level determining section 53 determines that the specified number of impulse noise components is 30 or more.

Finally, the discontinuity-level determining section 53 outputs the discontinuity level (high) corresponding to the determined results to the result output unit 23 as discontinuity-level determination results.

The discontinuity-level determination results output from the discontinuity-level determining section 53 are displayed in the display unit 12 by the result output unit 23. For example, in a region of the display unit 12 shown in FIG. 29 in which level determination results are displayed, a message, such as “• discontinuity level: high” may be displayed.

With this operation, analysis results of body sound information collected by a stethoscope can be provided to a user such that they are easy to understand. That is, not only results indicating whether body sounds are normal or abnormal, but also, if body sounds are abnormal, the degree (level) of the abnormality can be provided to a user such that it is easy to understand.

### [Information Analyzing Processing Flow]

FIG. 30 is a flowchart illustrating a flow of information analyzing processing performed by the information analyzing apparatus 100 of this embodiment.

First, the body sound obtaining unit 20 obtains body sound information to be subjected to information analyzing processing from the digital stethoscope 3 via the communication unit 14 (S1).

Then, the body sound processor 21 processes a sound waveform included in the body sound information obtained by the body sound obtaining unit 20 so as to generate waveform feature information (S2).

Generating of waveform feature information by the body sound processor 21 in S2 includes: finding an autocorrelation function (waveform feature information) from a sound waveform by the autocorrelation analyzer 211; finding a spectrum (waveform feature information) from a sound waveform by the Fourier transform unit 212, finding a spectrogram (waveform feature information) from a sound waveform by the time-frequency analyzer 213; detecting an envelope (waveform feature information) of a sound waveform by the envelope detector 214; and specifying impulse noise of a sound waveform and outputting impulse noise detection results (waveform feature information) by the impulse noise detector 215. However, generating of waveform feature information is not restricted to these operations. Additionally, the body sound processor 21 may generate all of the above-described items of waveform feature information or only some of the items of waveform feature information.

Then, the waveform feature determining unit 30 analyzes the waveform feature information generated by the body sound processor 21, determines features of a sound waveform, and then generates waveform feature determination results reflecting the determined features (S3).

Generating of waveform feature determination results by the waveform feature determining unit 30 in S3 includes: executing determination item 1 or determination item 1′ and determining features as to the periodicity of body sounds by the periodicity determining section 31; executing determination item 2-A or determination item 2-B and determining features as to the frequency component distribution of body sounds by the spectrum determining section 32; executing determination item 3-A or determination item 3-B and determining features as to the periodicity of a time-frequency component distribution of body sounds by the spectrogram determining section 33; executing determination item 4 and determining features as to the continuity of adventitious sounds included in body sounds by the envelope determining section 34; and executing determination item 5 and determining features as to the discontinuity of adventitious sounds included in body sounds by the impulse noise determining section 35. However, generating of waveform feature determination results is not restricted to these operations. The waveform feature determining unit 30 may perform all of the above-described determination items or only some of the determination items.

For example, if waveform feature determination results concerning determination item 4 executed by the envelope determining section 34 indicate true, they can be sufficient grounds to determine by the continuous-adventitious-sound determining section 43 that “there is a possibility that subject breath sounds are continuous adventitious sounds”.

Accordingly, the following configuration is also encompassed in the invention of this application. The envelope determining section 34 of the waveform feature determining unit 30 executes determination item 4, and the continuous-adventitious-sound determining section 43 of the sound-type determining unit 40 determines whether or not breath sounds are continuous adventitious sounds, only on the basis of the waveform feature determination results concerning determination item 4.

Alternatively, for example, if waveform feature determination results concerning determination item 5 executed by the impulse noise determining section 35 indicate true, they can be sufficient grounds to determine by the discontinuous-adventitious-sound determining section 44 that “there is a possibility that subject breath sounds are discontinuous adventitious sounds”.

Accordingly, the following configuration is also encompassed in the invention of this application. The impulse noise determining section 35 of the waveform feature determining unit 30 executes determination item 5, and the discontinuous-adventitious-sound determining section 44 of the sound-type determining unit 40 determines whether or not breath sounds are discontinuous adventitious sounds, only on the basis of the waveform feature determination results concerning determination item 5.

Then, the sound-type determining unit 40 determines a sound type of sound waveform on the basis of the waveform feature determination results generated by the waveform feature determining unit 30, and generates sound-type determination results reflecting the determined sound type (S4).

Generating of sound-type determination results by the sound-type determining unit 40 in S4 includes: determining whether or not the body sounds are normal breath sounds by the normal-breath-sound determining section 41; determining whether or not the body sounds are decreased breath sounds by the decreased-breath-sound determining section 42; determining whether or not the body sounds are continuous adventitious sounds by the continuous-adventitious-sound determining section 43; and determining whether or not the body sounds are discontinuous adventitious sounds by the discontinuous-adventitious-sound determining section 44. However, generating of sound-type determination results is not restricted to these operations. The sound-type determining unit 40 may perform determination concerning all of the above-described sound types or may perform determination concerning only some of the sound types.

If the body sound analyzer 22 does not include the abnormality-level determining unit 50, or if the sound-type determining unit 40 has not classified a sound type of body sounds as abnormal sounds (1 in S5), S6 is executed, and the information analyzing apparatus 100 terminates the information analyzing processing. That is, the result output unit 23 displays sound-type determination results output from the sound-type determining unit 40 in the display unit 12 (S6).

For example, in S6, as shown in FIG. 29, the result output unit 23 displays sound-type determination results output from the individual determining sections of the sound-type determining unit 40 in a region of the display unit 12 in which analysis results are displayed.

If the sound-type determining unit 40 has determined that there is a possibility that body sounds are abnormal sounds (in this case, decreased breath sounds, continuous adventitious sounds, or discontinuous adventitious sounds), it may count the frequency with which such abnormal sounds have appeared in the body sounds. Then, the result output unit 23 may also display the frequency of appearances of such abnormal sounds in a region in which analysis results are displayed.

If the body sound analyzer 22 includes the abnormality-level determining unit 50, and if the sound-type determining unit 40 has classified a sound type of body sounds as abnormal sounds (2 in S5), the abnormality-level determining unit 50 determines the level of the abnormality. The abnormality-level determining unit 50 determines the degree of the abnormality of the classified sound type and generates abnormality-level determination results (S7).

Generating of abnormality-level determination results by the abnormality-level determining unit 50 in S7 includes: determining a decreased sound level from a spectrogram and generating decreased-sound-level determination results by the decreased-sound-level determining section 51; determining a continuity level from an envelope and generating continuity-level determination results by the continuity-level determining section 52; and determining a discontinuity level from impulse noise detection results and generating discontinuity-level determination results by the discontinuity-level determining section 53. However, generating of abnormality-level determination results is not restricted to these operations. The abnormality-level determining unit 50 may perform level determination concerning all of the above-described types of abnormal sounds or may perform level determination concerning only some of the types of abnormal sounds.

Finally, the result output unit 23 displays sound-type determination results output from the sound-type determining unit 40 and abnormality-level determination results output from the abnormality-level determining unit 50 in the display unit 12 (S8). For example, as shown in FIG. 29, the result output unit 23 displays a value, such as “low”, “intermediate”, or “high”, indicating an abnormality level, in a region in which abnormality-level determination results are displayed, according to the type of abnormal sound.

In this embodiment, as shown in FIG. 22, the normal-breath-sound determining section 41 determines whether or not breath sounds are normal, on the basis of waveform feature determination results concerning determination item 1, determination item 2-A, and determination item 3-A output from the corresponding determining sections of the waveform feature determining unit 30. However, the normal-breath-sound determining section 41 of the present invention is not restricted to this configuration.

For example, the decreased-breath-sound determining section 42 of the sound-type determining unit 40 may determine whether or not there is a possibility that breath sounds are decreased breath sounds, the continuous-adventitious-sound determining section 43 of the sound-type determining unit 40 may determine whether or not there is a possibility that breath sounds are continuous adventitious sounds, and the discontinuous-adventitious-sound determining section 44 of the sound-type determining unit 40 may determine whether or not there is a possibility that breath sounds are discontinuous adventitious sounds. Then, if the breath sounds are not determined to be any of the abnormal sounds, the normal-breath-sound determining section 41 may determine whether the breath sounds are (may be) normal.

### Second Embodiment

Another embodiment of an information analyzing apparatus of the present invention will be described below with reference to FIGS. 31 through 37. For the convenience of description, elements having the same functions as those shown in the drawings discussed in the above-described first embodiment are designated by like reference numerals, and an explanation thereof will thus be omitted.

In the above-described first embodiment, the sound-type determining unit 40 includes individual sound-type determining sections for making a determination as to sound types to be classified whether or not body sounds are of such sound types.

However, the information analyzing apparatus 100 of the present invention is not restricted to this configuration.

Instead of including determining sections according to the sound types, the sound-type determining unit 40 may include a comprehensive determination section 45 that performs a comprehensive determination on the basis of all features of body sounds so that the body sounds can be classified as a single sound type.

In the configuration in which multiple determining sections are provided according to the sound types, there may be inconsistencies among multiple determination results. In the above-described configuration, however, body sounds are always classified as a single sound type. Accordingly, it is possible to provide determination results which are easier to understand for a user.

### [Functional Configuration of Information Analyzing Apparatus]

FIG. 31 is a functional block diagram illustrating the major configuration of the information analyzing apparatus 100 of this embodiment.

The information analyzing apparatus 100 shown in FIG. 31 is different from that shown in FIG. 1 in that the sound-type determining unit 40 does not include the normal-breath-sound determining section 41, the decreased-breath-sound determining section 42, the continuous-adventitious-sound determining section 43, and the discontinuous-adventitious-sound determining section 44, but includes the comprehensive determination section 45.

The comprehensive determination section 45 specifies a sound type of subject body sound by comprehensively using waveform feature determination results output from individual determining sections of the waveform feature determining unit 30.

The functional blocks of the above-described controller 10, in particular, the comprehensive determination section 45, are implemented as a result of, for example, a CPU (central processing unit), reading a program stored in a storage device (storage unit 13) implemented by, for example, a ROM (read only memory) or an NVRAM (non-volatile random access memory) into, for example, a RAM (random access memory), and executing the read program.

### (Comprehensive Determination Function)

FIG. 32 is a diagram illustrating a sound type system used by the comprehensive determination section 45 of this embodiment for classifying respiratory system sounds obtained from a patient P as a predetermined sound type. As shown in FIG. 32, in this embodiment, the comprehensive determination section 45 classifies respiratory system sounds as one of “normal breath sounds”, “decreased breath sounds”, “other abnormal sounds”, “high-pitched continuous adventitious sounds”, “low-pitched continuous adventitious sounds”, “fine discontinuous adventitious sounds”, “coarse discontinuous adventitious sounds”, and “other adventitious sounds”. Then, the comprehensive determination section 45 outputs a specified sound type to the result output unit 23 as comprehensive determination results.

The comprehensive determination section 45 first classifies respiratory system sounds collected from the patient P into breath sounds and adventitious sounds. The comprehensive determination section 45 performs this classification on the basis of waveform feature determination results concerning determination item 1-1 and determination item 1-2 shown in FIG. 7 output from the periodicity determining section 31.

The comprehensive determination section 45 then classifies breath sounds into breath sounds (normal sounds or decreased sounds) and other abnormal sounds. The comprehensive determination section 45 performs this classification on the basis of waveform feature determination results concerning determination item 2-A shown in FIG. 11 output from the spectrum determining section 32.

The comprehensive determination section 45 then classifies breath sounds (normal sounds or decreased sounds) into normal breath sounds and decreased breath sounds. The comprehensive determination section 45 performs this classification on the basis of waveform feature determination results concerning determination item 3-A shown in FIG. 16 output from the spectrogram determining section 33.

The comprehensive determination section 45 then classifies adventitious sounds into continuous adventitious sounds and adventitious sounds other than continuous adventitious sounds. The comprehensive determination section 45 performs this classification on the basis of waveform feature determination results concerning determination item 4 shown in FIG. 18 output from the envelope determining section 34.

The comprehensive determination section 45 then classifies continuous adventitious sounds into high-pitched continuous adventitious sounds and low-pitched continuous adventitious sounds. The comprehensive determination section 45 performs this classification on the basis of waveform feature determination results concerning determination item 2-B shown in FIG. 11 output from the spectrum determining section 32.

The comprehensive determination section 45 then classifies adventitious sounds other than continuous adventitious sounds into discontinuous adventitious sounds and other adventitious sounds. The comprehensive determination section 45 performs this classification on the basis of waveform feature determination results concerning determination item 5 shown in FIG. 21 output from the impulse noise determining section 35.

The comprehensive determination section 45 then classifies discontinuous adventitious sounds into fine discontinuous adventitious sounds and coarse discontinuous adventitious sounds. The comprehensive determination section 45 performs this classification on the basis of waveform feature determination results concerning determination item 2-B shown in FIG. 11 output from the spectrum determining section 32.

### [Information Analyzing Processing Flow]

FIGS. 33A and 33B show a flowchart of a flow of information analyzing processing performed by the information analyzing apparatus 100 of this embodiment. In this embodiment, it is assumed that S1 and S2 in FIG. 30 have already been executed prior to S101 of FIG. 33A.

Upon completion of processing on body sounds by the body sound processor 21, the periodicity determining section 31 executes determination item 1-1 (S101). That is, the periodicity determining section 31 determines whether or not the waveform of an autocorrelation function has peaks at intervals of two to five seconds. The periodicity determining section 31 also executes determination item 1-2 (S102). That is, the periodicity determining section 31 determines whether a peak width (duration) with respect to the amplitude value at a position of ¼ of a peak amplitude value in the envelope of the autocorrelation function is 10% or smaller of the breathing period. The periodicity determining section 31 may execute either one of S101 or S102 first.

If both of determination item 1-1 and determination item 1-2 are true, that is, if the periodicity of body sounds is strong (YES in S103), the comprehensive determination section 45 classifies the body sounds as breath sounds (no adventitious sounds) (S104). Conversely, if at least one of determination item 1-1 and determination item 1-2 is false, that is, if the periodicity of the body sounds are weak (NO in S103), the comprehensive determination section 45 classifies the body sounds as adventitious sounds (S105).

Then, the spectrum determining section 32 executes determination item 2-A on the body sounds classified as breath sounds (S106). That is, the spectrum determining section 32 determines whether or not the total frequency components at 200 Hz or lower occupies 80% or higher of all frequency components.

If determination item 2-A is true, that is, if the frequency component distribution of the body sounds is substantially normal (YES in S107), the comprehensive determination section 45 classifies the body sounds as one of normal breath sounds and decreased breath sounds (S108). Conversely, if determination item 2-A is false, that is, if the frequency component distribution of the body sounds is likely to be abnormal (NO in S107), the comprehensive determination section 45 classifies the body sounds as other adventitious sounds (S109).

Then, the spectrogram determining section 33 executes determination item 3-A on the body sounds classified as one of normal breath sounds and decreased breath sounds (S110). That is, the spectrogram determining section 33 determines whether or not a strong periodicity of frequency components is observed in a range of 400 Hz (or higher).

If determination item 3-A is true, that is, if frequency components of breath sounds are observed in a high frequency range of body sounds (YES in S111), the comprehensive determination section 45 classifies the body sounds as normal breath sounds (S112). Conversely, if determination item 3-A is false, that is, if frequency components of breath sounds are not observed in a high frequency range of body sounds (NO in S111), the comprehensive determination section 45 classifies the body sounds as decreased breath sounds (S113). If the body sound analyzer 22 includes the decreased-sound-level determining section 51, the decreased-sound-level determining section 51 determines the decreased sound level of the body sounds (S114).

On the other hand, if the comprehensive determination section 45 classifies the body sounds as adventitious sounds, as shown in FIG. 33B, the envelope determining section 34 executes determination item 4 on the body sounds classified as adventitious sounds (S115). That is, the envelope determining section 34 determines whether or not the continuity is observed in the envelope (adventitious sounds).

If determination item 4 is true, that is, if the continuity is observed in the adventitious sounds of the body sounds (YES in S116), the comprehensive determination section 45 classifies the body sounds as continuous adventitious sounds (S117).

Then, the spectrum determining section 32 executes determination item 2-B on the body sounds classified as continuous adventitious sounds (S118). That is, the spectrum determining section 32 determines whether or not the total frequency components at 200 Hz or higher occupies 30% or higher of all frequency components.

If determination item 2-B is true, that is, if relatively many frequency components in a high frequency range are observed (YES in S119), the comprehensive determination section 45 classifies the body sounds as high-pitched continuous adventitious sounds (S120). Conversely, if determination item 2-B is false, that is, if many frequency components in a high frequency range are not observed (NO in S119), the comprehensive determination section 45 classifies the body sounds as low-pitched continuous adventitious sounds (S121). If the body sound analyzer 22 includes the continuity-level determining section 52, the continuity-level determining section 52 determines the continuity level of the body sounds (S122).

If determination item 4 is false in S116, that is, if the continuity is not observed in the adventitious sounds of the body sounds (NO in S116), the comprehensive determination section 45 classifies the body sounds as adventitious sounds other than continuous adventitious sounds (S123).

Then, the impulse noise determining section 35 executes determination item 5 on the body sounds classified as adventitious sounds other than continuous adventitious sounds (S124). That is, the impulse noise determining section 35 determines whether or not the number of impulse noise components per period is ten or more.

If determination item 5 is true, that is, if the discontinuity is observed in the adventitious sounds of the body sounds (YES in S125), the comprehensive determination section 45 classifies the body sounds as discontinuous adventitious sounds (S126). Conversely, if determination item 5 is false, that is, if the discontinuity is not observed in adventitious sounds of the body sounds (NO in S125), the comprehensive determination section 45 classifies the body sounds as other adventitious sounds (S127).

Then, the spectrum determining section 32 executes determination item 2-B on the body sounds classified as discontinuous adventitious sounds (S128). That is, the spectrum determining section 32 determines whether or not the total frequency components at 200 Hz or higher occupies 30% or higher of all frequency components.

If determination item 2-B is true, that is, if relatively many frequency components in a high frequency range are observed (YES in S129), the comprehensive determination section 45 classifies the body sounds as fine discontinuous adventitious sounds (S130). Conversely, if determination item 2-B is false, that is, if many frequency components in a high frequency range are not observed (NO in S129), the comprehensive determination section 45 classifies the body sounds as coarse discontinuous adventitious sounds (S131). If the body sound analyzer 22 includes the discontinuity-level determining section 53, the discontinuity-level determining section 53 determines the discontinuity level of the body sounds (S132).

Finally, as shown in FIG. 33A, the result output unit 23 displays comprehensive determination results, output from the comprehensive determination section 45, indicating one of the above-described sound types as which the body sounds are classified in the display unit 12 (S133). If the abnormality-level determining unit 50 outputs abnormality-level determination results, the result output unit 23 also displays the abnormality-level determination results in the display unit 12. [Level Determining Processing Flow]

Flows of abnormality-level determining processing performed by the individual determining sections of the abnormality-level determining unit 50 will be described below with reference to FIGS. 34 through 36. The processing flows of the individual determining sections of the abnormality-level determining unit 50 shown in FIGS. 34 through 36 are used both for the first embodiment and the second embodiment.

FIG. 34 is a flowchart illustrating a flow of decreased-sound-level determining processing performed by the decreased-sound-level determining section 51.

When decreased-sound-level determining processing is started in S7 of FIG. 30 or S114 of FIG. 33A, the decreased-sound-level determining section 51 first scans a spectrogram of subject body sounds and specifies the frequency at a boundary between a frequency range in which the periodicity is strong (observed) and a frequency range in which the periodicity is weak (is not observed) (S201). Then, the decreased-sound-level determining section 51 refers to the decreased-sound-level determination criteria shown in FIG. 26 stored in the storage unit 13.

If the frequency at the above-described boundary is in a range from 300 Hz to 400 Hz (YES in S202), the decreased-sound-level determining section 51 determines that the decreased sound level is low (S203).

If the frequency at the above-described boundary is not in a range from 300 Hz to 400 Hz (NO in S202), the decreased-sound-level determining section 51 further determines whether or not the frequency at the above-described boundary is in a range from 200 Hz to lower than 300 Hz (S204). Then, if the frequency at the above-described boundary is in a range from 200 Hz to lower than 300 Hz (YES in S204), the decreased-sound-level determining section 51 determines that the decreased sound level is intermediate (S205).

If the frequency at the above-described boundary is not in a range from 200 Hz to lower than 300 Hz (NO in S204), it means that the frequency at the boundary is lower than 200 Hz. In this case, the decreased-sound-level determining section 51 determines that the decreased sound level is high (S206).

The decreased-sound-level determination results output from the decreased-sound-level determining section 51 are output to the result output unit 23.

FIG. 35 is a flowchart illustrating a flow of continuity-level determining processing performed by the continuity-level determining section 52.

When continuity-level determining processing is started in S7 of FIG. 30 or S122 of FIG. 33B, the continuity-level determining section 52 first specifies a continuous time for which the amplitude exceeds the amplitude average value in an envelope of a sound waveform of subject body sounds (S301). Then, the continuity-level determining section 52 refers to, for example, the continuity-level determination criteria shown in FIG. 27 stored in the storage unit 13.

If the continuous time is in a range from 200 ms to shorter than 600 ms (YES in S302), the continuity-level determining section 52 determines that the continuity level is low (S303).

If the continuous time is not in a range from 200 ms to shorter than 600 ms (NO in S302), the continuity-level determining section 52 further determines whether or not the continuous time is in a range from 600 ms to shorter than 1000 ms (S304). Then, if the continuous time is from 600 ms to shorter than 1000 ms (YES in S304), the continuity-level determining section 52 determines that the continuity level is intermediate (S305).

If the continuous time is not in a range from 600 ms to shorter than 1000 ms (NO in S304), it means that the continuous time is 1000 ms or longer. In this case, the continuity-level determining section 52 determines that the continuity level is high (S306).

The continuity-level determination results output from the continuity-level determining section 52 are output to the result output unit 23.

In the example shown in FIG. 35, the continuity level is determined on the basis of a continuous time for which the amplitude exceeds the amplitude average value. However, the continuity-level determining section 52 is not restricted to this configuration. For example, the continuity-level determining section 52 may determine the continuity level on the basis of a total time for which the amplitude exceeds the amplitude average value in an envelope per period.

FIG. 36 is a flowchart illustrating a flow of discontinuity-level determining processing performed by the discontinuity-level determining section 53.

When discontinuity-level determining processing is started in S7 of FIG. 30 or S132 of FIG. 33B, the discontinuity-level determining section 53 first specifies the number of impulse noise components per period in a waveform of subject body sounds (S401). Then, the discontinuity-level determining section 53 refers to, for example, the discontinuity-level determination criteria shown in FIG. 28, stored in the storage unit 13.

If the number of impulse noise components is ten to less than twenty (YES in S402), the discontinuity-level determining section 53 determines that the discontinuity level is low (S403).

If the number of impulse noise components is not ten to less than twenty (NO in S402), the discontinuity-level determining section 53 further determines whether or not the number of impulse noise components is twenty to less than thirty (S404). Then, if the number of impulse noise components is twenty to less than thirty (YES in S404), the discontinuity-level determining section 53 determines that the discontinuity level is intermediate (S405).

If the number of impulse noise components is not twenty to less than thirty (NO in S404), it means that the number of impulse noise components is thirty or more. In this case, the discontinuity-level determining section 53 determines that the discontinuity level is high (S406).

The discontinuity-level determination results output from the discontinuity-level determining section 53 are output to the result output unit 23.

### [Result Output Function]

As discussed above, the result output unit 23 displays, in the display unit 12, comprehensive determination results output from the comprehensive determination section 45, indicating one of the above-described sound types as which the body sounds are classified. For example, as shown in FIG. 37, the result output unit 23 displays the comprehensive determination results in a region in which analysis results are displayed. In FIG. 37, an example of comprehensive determination results indicating that the comprehensive determination section 45 has classified body sounds as high-pitched continuous adventitious sounds is shown. If the comprehensive determination section 45 counts the frequency of appearances of such abnormal sounds in the waveform of the body sounds, the result output unit 23 may also display the frequency of appearances obtained from the comprehensive determination section 45 in the display unit 12.

If the abnormality-level determining unit 50 outputs abnormality-level determination results, the result output unit 23 may also display the abnormality-level determination results in the display unit 12. In the example shown in FIG. 37, the body sounds are classified as high-pitched continuous adventitious sounds. Accordingly, the result output unit 23 displays continuity-level determination results determined by the continuity-level determining section 52 in a region in which level determination results are displayed.

The result output unit 23 may display a “play back sound” button, as shown in FIGS. 29 and 37, and receive an instruction to play back body sounds subjected to analysis processing from the operator U.

For example, if the operator U performs single tapping on the “play back sound” button, the result output unit 23 may play back body sound information obtained by the body sound obtaining unit 20 and output a sound signal to a sound output unit (not shown). If the operator U performs double tapping on the “play back sound” button, the result output unit 23 may control the sound output unit so that sound can be played back from a portion at which an abnormality appears in the body sounds.

If the operator U taps a “store sound and results” button, the result output unit 23 stores the above-described body sound information, determination results, and necessary information concerning a patient in association with each other in the storage unit 13.

If the operator U taps the “store sound and results” button, the result output unit 23 may store the body sound information associated with determination results in a database (not shown) of an external device. More specifically, the result output unit 23 may send various determination results received from the body sound analyzer 22, together with collected body sound information, to an external device via the communication unit 14. For example, the communication unit 14 of the information analyzing apparatus 100 is able to send determination results and body sound information to the management server 4 via the communication network 5.

With this operation, the management server 4 is able to display the determination results shown in FIG. 29 or 37 in a display unit of the management server 4 and to provide the determination results concerning the body sounds of the patient P to the physician D located in a remote site. The management server 4 is also able to, in response to an instruction from the physician D, play back body sound information desired by the physician D and to allow the physician D to listen to the body sound information.

With the above-described configuration and method, the body sound processor 21 processes body sound information and extracts waveform feature information from a sound waveform. The waveform feature determining unit 30 then determines which determination criterion the waveform feature information matches (or does not match). The comprehensive determination section 45 is then able to specify the type of body sound on the basis of determination results of the waveform features. More specifically, the comprehensive determination section 45 is able to classify the body sounds as the most likely sound type among a plurality of types which are defined in advance based on medical features in terms of sounds.

Comprehensive determination results obtained by the comprehensive determination section 45 are displayed in the display unit 12 as analysis results.

Concerning the above-described determination criteria, thresholds are defined in advance on the basis of medical features highly related to each sound type. Accordingly, depending on whether or not extracted waveform feature information matches the determination criteria, the comprehensive determination section 45 is able to determine with which sound type the original body sound information has a high (or low) correlation.

With this configuration, the type of body sound information can be specified without directly comparing it with model waveforms. Accordingly, it is possible to realize an information analyzing apparatus that highly precisely and efficiently conducts objective analyses without depending on the completeness of a model sound waveform database and that provides analysis results to a user.

### Modified Example

In the above-described first and second embodiments, in the auscultation system 200 of the present invention, the function of analyzing information concerning, for example, breath sounds, is implemented by the information analyzing apparatus 100, which serves as a terminal device operated by the operator U. In the above-described first and second embodiments, in the auscultation system 200, the information analyzing apparatus 100 communicates with the digital stethoscope 3 and the management server 4 in the support center 2.

However, the auscultation system 200 of the present invention is not restricted to this configuration. In the auscultation system 200, the function of analyzing information concerning, for example, breath sounds, performed by the information analyzing apparatus 100 of the present invention may be mounted on the digital stethoscope 3 and/or the management server 4 in the support center 2. In this case, the digital stethoscope 3 and/or the management server 4 function as the information analyzing apparatus of the present invention.

### Third Embodiment

Another embodiment of the present invention will be described below with reference to FIG. 38. For the convenience of description, elements having the same functions as those shown in the drawings discussed in the above-described first and second embodiments are designated by like reference numerals, and an explanation thereof will thus be omitted.

### BACKGROUND ART AND PROBLEMS

PTL 3 discloses a medical image display system for creating and displaying a medical image in the following manner. A predetermined part of a body is imaged and image data indicating such an image part is obtained. Body sound measurement is then performed on the part of the body indicated in the image data. By associating measurement results of body sounds and the corresponding part of the body, a medical image is displayed.

In this configuration of the related art, however, imaging is performed without using measurement results of body sounds, and thus, it is not possible to perform imaging by focusing on a specific part in which an abnormality is occurring. Additionally, if there is no problem in the results of body sound measurement, the imaging operation performed on the body turns out to be useless.

Accordingly, in this embodiment, a measurement system which performs medical imaging by considering measurement results of body sounds will be discussed.

### [Overview of Measurement System]

FIG. 38 is a block diagram illustrating an overview of a measurement system 3600 according to a third embodiment and the major configuration of an imaging apparatus 3006 forming the measurement system 3600.

The measurement system 3600 includes at least the digital stethoscope 3 and the imaging apparatus 3006. The measurement system 3600 may also include the above-described auscultation system 200 (FIG. 2) if necessary. That is, if necessary, the digital stethoscope 3 and the imaging apparatus 3006 of the third embodiment are able to connect to various devices within the auscultation system 200 in the above-described first and second embodiments so that they can communicate with such devices, and to operate in cooperation with the auscultation system 200.

The digital stethoscope 3 collects body sound information of a patient P. In this embodiment, the digital stethoscope 3 is the digital stethoscope 3 which serves as part of the auscultation system 200 shown in FIG. 2.

The imaging apparatus 3006 images the patient P by using a suitable imaging unit so as to obtain image data. The image data obtained by the imaging apparatus 3006 is utilized by the operator U or the physician P as a medical image.

In this embodiment, the imaging apparatus 3006 is cooperated with the auscultation system 200 shown in FIG. 2. The imaging apparatus 3006 is able to select optimal imaging processing for the patient P by considering auscultation results of the patient P obtained by the auscultation system 200 and to perform the selected optimal imaging processing.

### [Configuration of Imaging Apparatus]

The imaging apparatus 3006 includes, as shown in FIG. 38, a communication unit 3011 which sends and receives information to and from the individual devices of the auscultation system 200, a storage unit 3012 which stores therein various items of information processed by the imaging apparatus 3006, an imaging unit 3013 which images a patient, and a controller 3010 which centrally controls the individual elements of the imaging apparatus 3006.

The communication unit 3011 communicates with the individual devices of the auscultation system 200 and receives auscultation results of the patient P obtained by the auscultation system 200.

The storage unit 3012 stores therein, for example, image data obtained by the imaging unit 3013 and analysis result information d1 and body part information d2 obtained by the communication unit 3011.

The imaging unit 3013 images a body by using suitable means such as X rays, CT (Computed Tomography), MRI (Magnetic Resonance Imaging), magnetic measurement, bioelectric signals, ultrasound, or light, though the suitable means is not restricted thereto. In order to image a desired part of a patient P, the imaging unit 3013 may include a positioning mechanism for positioning an image sensor to an appropriate body part.

The controller 3011 includes, as functional blocks, an auscultation-result obtaining section 3020, an imaging-part specifying section 3021, and an imaging control section 3022.

The auscultation-result obtaining section 3020 controls the communication unit 3011 so that it can obtain auscultation results from the information analyzing apparatus 100. Auscultation results obtained by the auscultation-result obtaining section 3020 include two types of information. One type is analysis result information d1 indicating analysis results concerning body sound information collected by the digital stethoscope 3. The other type is body part information d2 indicating a body part from which the body sound information is obtained. Specifically, the auscultation-result obtaining section 3020 obtains auscultation results at least indicating the presence or the absence of an abnormality, which has been determined on the basis of the body sound information concerning the patient P by the information analyzing apparatus 100, and a body part from which the body sound information has been collected.

More specifically, in this embodiment, the imaging apparatus 3006 is connected to the information analyzing apparatus 100 of the first or second embodiment so that it can communicate with the information analyzing apparatus 100. The auscultation-result obtaining section 3020 obtains, from the information analyzing apparatus 100 of the first embodiment via the communication unit 3011, sound-type determination results determined by the sound-type determining unit 40, and in some cases, level determination results determined by the abnormality-level determining unit 50, as the analysis result information d1. Alternatively, the auscultation-result obtaining section 3020 obtains, from the information analyzing apparatus 100 of the second embodiment via the communication unit 3011, comprehensive determination results determined by the comprehensive determination section 45, and in some cases, level determination results determined by the abnormality-level determining unit 50, as the analysis result information d1.

In this embodiment, it is assumed that, when body sound information is stored in and managed by the information analyzing apparatus 100 or the management server 4 in the auscultation system 200, body part information indicating a body part from which the body sound information has been collected is associated with the body sound information. The information analyzing apparatus 100 may receive input of body part information immediately before the operator U collects body sound information from the patient P by using the digital stethoscope 3. The operator U may broadly input “lungs”, or in more details, such as “right lung” or “left lung”, or even more details, such as “right upper lobe”, “right middle lobe”, “right lower lobe”, “left upper lobe”, or “left lower lobe”. Alternatively, as shown in FIG. 39, lungs may be divided into some portions and the divided portions are defined on the basis of the diameter of a tracheal. In this case, the operator U may input “shallow portion” indicating the upper part A of a tracheal (respiratory tract) which does not branch off into a deep level, or a relatively thin portion of the respiratory tract (a smaller-diameter portion of the tracheal), that is, “deep portion” indicating the lower part B of the tracheal (respiratory tract) which branches off into a deep level. In this manner, the body part specified by the operator U is associated with body sound information and is stored in the information analyzing apparatus 100. For example, a body part linking unit (not shown) of the information analyzing apparatus 100 links body part information input from the input unit 11 to analysis results output from the body sound analyzer 22. The result output unit 23 of the information analyzing apparatus 100 sends the body part information linked to the body sound information, as body part information d2, together with analysis result information d1 concerning the body sound information, to the imaging apparatus 3006.

The auscultation-result obtaining section 3020 obtains auscultation results which have been sent as described above, that is, the analysis result information d1 and the body part information d2. The auscultation results obtained by the auscultation-result obtaining section 3020 are utilized for specifying a body part to be imaged by the imaging-part specifying section 3021.

The imaging-part specifying section 3021 specifies a body part to be imaged by the imaging unit 3013. The imaging-part specifying section 3021 specifies, as a part to be imaged, a position at which body sound information indicating the occurrence of abnormality or possible abnormality suggested by the analysis result information d1 has been collected. The imaging-part specifying section 3021 is able to specify a part to be imaged by using the body part information d2 obtained together with the analysis result information d1.

For example, it is assumed that the analysis result information d1 obtained from the information analyzing apparatus 100 of the first embodiment includes at least one of sound-type determination results indicating “there is a possibility that body sounds are not normal breath sounds”, “there is a possibility that body sounds are decreased breath sounds”, “there is a possibility that body sounds are continuous adventitious sounds”, and “there is a possibility that body sounds are discontinuous adventitious sounds”. In this case, the imaging-part specifying section 3021 refers to the body part information d2 obtained together with the analysis result information d1 so as to specify a part to be imaged. For example, if the body part information d2 indicates “left lower lobe”, the imaging-part specifying section 3021 specifies “left lower lobe” as a part to be imaged since there is a sign of abnormality in “left lower lobe”. Alternatively, it is assumed that the analysis result information d1 obtained from the information analyzing apparatus 100 of the second embodiment includes comprehensive determination results indicating a certain type of abnormality other than “there is a high possibility that body sounds are normal breath sounds”. In this case, too, the imaging-part specifying section 3021 refers to the body part information d2 obtained together with the analysis result information d1 so as to specify a part to be imaged.

The imaging-part specifying section 3021 may be used, not only for selecting a part to be subjected to imaging, but also for refining a part to be subjected to precise imaging with higher resolution. For example, the imaging-part specifying section 3021 may determine that only “left lower lobe” exhibiting a sign of abnormality will be imaged with a setting (for example, with higher resolution) different from a regular setting for the other parts.

The imaging control section 3022 sets various settings for the imaging unit 3013 on the basis of the body part specified by the imaging-part specifying section 3021, and then controls the imaging unit 3013 so that the body will be imaged. That is, the imaging control section 3022 performs imaging processing so that settings (imaging techniques) for the part specified by the imaging-part specifying section 3021 will be different from those for the other parts.

For example, if the part specified by the imaging-part specifying section 3021 is “left lower lobe”, the imaging control section 3022 controls a positioning mechanism of the imaging unit 3013 so that the left lower lobe of the patient P will be precisely imaged. Alternatively, the imaging control section 3022 may set settings for the imaging unit 3013 so that imaging will be performed with higher precision only for the left lower lobe, and then perform imaging on the left lower lobe and the other parts.

Image data obtained by the imaging unit 3013 under the control of the imaging control section 3022 is stored in the storage unit 3012. In this case, when storing the image data, the imaging control section 3022 preferably associates the obtained image data with the corresponding analysis result information d1 and body part information d2. For example, the imaging control section 3022 associates the image data obtained by imaging the left lower lobe by the imaging unit 3013 with the analysis result information d1 indicating “there is a possibility that body sounds are continuous adventitious sounds” and the body part information d2 indicating “left lower lobe” and stores the image data in the storage unit 3012.

If a device including a function of analyzing body sound information and determining a disease is included in the auscultation system 200, the analysis result information d1 may include information concerning the name of a disease if necessary. By informing the imaging apparatus 3006 of the name of a disease, the imaging control section 3022 is able to associate the name of a possible disease to obtained image data and store the image data in the storage unit 3012. If such image data is displayed in a display unit (not shown) together with the name of a disease and sound-type determination results, more detailed information can be provided to the physician D.

On the other hand, there may be some cases in which supplying of the degree (level) of abnormality to the imaging apparatus 3006 is more preferable than supplying of the name of a disease, as analysis result information d1. The reason for this is as follows. In the imaging apparatus 3006 of the present invention, it is possible to restrict parts of the patient P to be subjected to imaging processing to a minimal level. In this case, if the level of abnormality (the above-described abnormality determination results) occurring in the patient P is supplied to the imaging apparatus 3006 as the analysis result information d1, the imaging-part specifying section 3021 is able to specify a part to be imaged in more details in accordance with the level of abnormality. More specifically, the imaging-part specifying section 3021 is able to specify the size of an area to be imaged in accordance with the level of abnormality. Although imaging of a medical image with an unnecessarily large size is preferably avoided, an image size which is not sufficient to provide necessary information for a physician D to examine a patient P is pointless. Accordingly, it is desirable that, as auscultation results, in addition to body part information d2 indicating an abnormal part, analysis result information d1 indicating analysis results including the level of abnormality is supplied to the imaging apparatus 3006. Then, the imaging-part specifying section 3021 of the imaging apparatus 3006 preferably specifies the size of an area to be imaged in accordance with the level of abnormality.

The imaging control section 3022 controls the imaging unit 3013 in accordance with the size specified by the imaging-part specifying section 3021 so that it can obtain a medical image concerning a suitable part with a suitable size.

Hitherto, when imaging a medical image, it is necessary that the operator U (or the physician D) of the imaging apparatus 3006 decide a part of a subject person (patient P) to be measured and operate the imaging apparatus 3006 so as to measure this part. In the measurement system 3600 of the present invention, on the basis of a part to be imaged specified by the imaging-part specifying section 3021 and the size of an area to be imaged determined by the imaging-part specifying section 3021, the imaging control section 3022 is able to position the imaging unit 3013 to a suitable location with respect to the subject person and to obtain a medical image. The obtained image data is then associated with body part information d2 and analysis result information d1 (the type and the level of abnormality) and is stored in the storage unit 3012. The stored image data is utilized as a medical image for conducting diagnosis by the physician D. Additionally, by managing the above-described attachment information associated with the image data, when reimaging of the same subject person becomes necessary after the first auscultation, the attachment information can be used as reference information. This also makes it possible to enhance the measurement precision in subsequent imaging processing. For example, the above-described attachment information can be utilized as follows. There may be a case in which a medical image measured for the first time does not have information that the physician D has expected (the resolution is low, the imaging area is small, or an abnormal part has not been properly imaged). In this case, the imaging-part specifying section 3021 may make corrections by changing the part to be measured, the resolution, or the size of an area to be imaged from those specified in the previous measurement so that image data having information desired by the physician D can be obtained.

As described above, in the measurement system 3600 of the present invention, the imaging apparatus 3006 is able to restrict parts of a patient P to be subjected to imaging processing to a minimal level by considering auscultation results output from the auscultation system 200. That is, it is possible to implement the imaging apparatus 3006 and an imaging method that are capable of performing imaging processing which can provide sufficient information for a physician D to conduct diagnosis and which can also minimize the burden on a patient P. More specifically, the imaging-part specifying section 3021 is able to decide to perform imaging, on the basis of auscultation results, only on a part in which the occurrence of abnormality (or possible abnormality) is recognized, or to perform imaging only on this part with higher resolution. For example, if the imaging unit 3013 is a mechanism which performs imaging with X rays, it is possible to reduce the radiation dose to which the patient P is exposed.

The present invention is not restricted to the above-described embodiments, and various modifications and changes may be made within the scope of the claims. An embodiment obtained by suitably combining technical means disclosed in the different embodiments is also encompassed in the technical scope of the present invention.

In the above-described embodiments, an example in which the information analyzing apparatus 100 of the present invention is applied to a smartphone has been discussed. However, the information analyzing apparatus 100 of the present invention may be implemented in the form of various information processing apparatuses. For example, the information analyzing apparatus 100 of the present invention is applicable to a personal computer (PC), an AV machine, such as a digital television, a notebook personal computer, a tablet PC, a cellular phone, and a PDA (Personal Digital Assistant), though it is not restricted thereto. The information analyzing apparatus 100 may be mounted on the digital stethoscope 3.

### [Examples of Implementations by Using Software]

The individual blocks of the information analyzing apparatus 100, in particular, the body sound obtaining unit 20, the body sound processor 21, the body sound analyzer 22, the result output unit 23, and the individual blocks of the body sound processor 21 and the body sound analyzer 22 may be implemented in the form of a hardware logic, or may be implemented in the form of software by using a CPU in the following manner.

Additionally, the individual blocks of the imaging apparatus 3006, in particular, the auscultation-result obtaining section 3020, the imaging-part specifying section 3021, and the imaging control section 3022 may be implemented in the form of a hardware logic, or may be implemented in the form of software by using a CPU in the following manner.

That is, the information analyzing apparatus 100 and the imaging apparatus 3006 each include a CPU (central processing unit) that executes commands of a control program which implements the individual functions, a ROM (read only memory) storing this program therein, a RAM (random access memory) loading this program, a storage device (recording medium), such as a memory, storing this program and various items of data therein, and so on. The object of the present invention may also be implemented by supplying a recording medium on which program code (an execution form program, an intermediate code program, and a source program) of the control program for each of the information analyzing apparatus 100 and the imaging apparatus 3006, which is software implementing the above-described functions, is recorded in a computer readable manner, to the information analyzing apparatus 100 and the imaging apparatus 3006, and by reading and executing the program code recorded on the recording medium by a computer (or a CPU or an MPU) of each of the information analyzing apparatus 100 and the imaging apparatus 3006.

As the above-described recording medium, for example, a tape type, such as magnetic tape or cassette tape, a disk type including a magnetic disk, such as a floppy (registered trademark) disk or a hard disk, and an optical disc, such as a CD-ROM, an MO, an MD, a DVD, or a CD-R, a card type, such as an IC card (including a memory card) or an optical card, or a semiconductor memory type, such as a mask ROM, an EPROM, an EEPROM (registered trademark), or a flash ROM may be used.

The information analyzing apparatus 100 and the imaging apparatus 3006 may be configured such that they are connectable to a communication network, and the above-described program code may be supplied to the information analyzing apparatus 100 and the imaging apparatus 3006 via the communication network. This communication network is not particularly restricted, and, for example, the Internet, an intranet, an extranet, a LAN, an ISDN, a VAN, a CATV communication network, a VPN (virtual private network), a public switched telephone network, a mobile communication network, a satellite communication work, etc. may be used. Additionally, a transmission medium forming this communication network is not restricted, and, for example, a wired transmission medium, such as IEEE1394, USB, power line communication, a cable TV line, a telephone line, or an ADSL circuit, or a wireless transmission medium, such as infrared, for example, IrDA or a remote controller, Bluetooth (registered trademark), 802.11 radio, HDR (High Data Rate), a cellular phone network, a satellite circuit, or a terrestrial digital network, may be used. The present invention may also be realized in the form of a computer data signal embedded in a carrier wave in which the above-described program code is implemented through digital transmission.

### SUMMARY

In order to solve the above-described problems, an information analyzing apparatus of the present invention includes: waveform feature determining means for applying waveform feature determination criteria to a sound waveform included in body sound information collected by a stethoscope so as to specify a feature of the sound waveform, the waveform feature determination criteria indicating criteria for classifying features of sound waveforms; and sound-type determining means for determining a sound type to which the body sound information belongs, on the basis of the feature of the sound waveform specified by the waveform feature determining means.

With this configuration, the waveform feature determining means is able to apply waveform feature determination criteria to a sound waveform included in body sound information so as to specify a feature of the sound waveform. Since the waveform feature determination criteria indicates criteria for classifying features of sound waveforms, the waveform feature determining means is able to always objectively classify a feature of any sound waveform in accordance with the waveform feature determination criteria.

The sound-type determining means is able to determine the type of sound included in the body sound information, on the basis of determination results obtained by the waveform feature determining means, that is, the classified type of specified feature. The sound-type determining means is able to highly precisely determine with which sound type the original body sound information has a high correlation, in accordance with the objective classification based on the waveform feature determination criteria.

With this configuration, the type of body sound information can be specified by analyzing a sound waveform itself of the body sound information in accordance with the waveform feature determination criteria, without directly comparing the waveform with model waveforms. Accordingly, it is possible to implement an information analyzing apparatus that highly precisely and efficiently conducts objective analyses without depending on the completeness of a model sound waveform database and that provides analysis results to a user.

In the information analyzing apparatus, the waveform feature determination criteria referred to by the waveform feature determining means may preferably include a threshold to be compared with a feature quantity found from the sound waveform and a condition determined by the threshold. The waveform feature determining means may preferably specify a feature of the sound waveform by determining whether or not the feature quantity of the sound waveform matches the condition.

In the waveform feature determination criteria, thresholds (quantitative values) are defined in advance on the basis of features highly related to the above-described sound types. Accordingly, the waveform feature determining means is able to determine whether or not the feature quantity extracted from the sound waveform matches the condition defined by the threshold and to supply determination results to the sound-type determining means. Then, the sound-type determining means is able to highly precisely determine, on the basis of the determination results, with which sound type the body sound information including this sound waveform has a high correlation.

With this configuration, a sound type can be specified efficiently and with stable precision merely by comparing a feature quantity extracted from body sound information with a threshold, without directly comparing a waveform of the body sound information with model waveforms. Accordingly, it is possible to implement an information analyzing apparatus that highly precisely and efficiently conducts objective analyses without depending on the completeness of a model sound waveform database and that provides analysis results to a user.

In the information analyzing apparatus, a sound type to be determined by the sound-type determining means may be at least one of: “normal breath sounds” indicating that breath sounds emitted from a living body are normal; “decreased breath sounds” indicating that breath sounds emitted from a living body are decreased before the breath sounds are collected by a stethoscope; “continuous adventitious sounds” indicating that breath sounds emitted from a living body include continuous adventitious sounds; and “discontinuous adventitious sounds” indicating that breath sounds emitted from a living body include discontinuous adventitious sounds.

With this configuration, the information analyzing apparatus is able to clarify to a user whether or not body sound information (breath sounds) collected by a stethoscope belongs to “normal breath sounds”. Alternatively, the information analyzing apparatus is able to clarify to a user whether or not body sound information (breath sounds) collected by a stethoscope belongs to “decreased breath sounds”. Alternatively, the information analyzing apparatus is able to clarify to a user whether or not body sound information (breath sounds) collected by a stethoscope belongs to “continuous adventitious sounds”. Alternatively, the information analyzing apparatus is able to clarify to a user whether or not body sound information (breath sounds) collected by a stethoscope belongs to “discontinuous adventitious sounds”.

The waveform feature determining means may determine, in accordance with of waveform feature determination criteria concerning an envelope, whether or not an envelope of a sound waveform continues with a certain or greater value of amplitude for a certain period or longer. If it is determined that the envelope continues with the certain or greater value of amplitude for the certain period or longer, the sound-type determining means may determine that there is a possibility that the body sound information belongs to continuous adventitious sounds.

If an envelope of a sound waveform of body sound information (breath sounds) collected by a stethoscope continues with a certain or greater value of amplitude for a certain period or longer, it may mean that adventitious sounds other than expiration sounds and inspiration sounds are continuously being generated. Accordingly, on the basis of a feature of the continuity of the envelope of the sound waveform, if the envelope continues with a certain or greater value of amplitude for a certain period or longer, the sound-type determining means can classify body sound information having such an envelope as continuous adventitious sounds.

With this configuration, concerning body sound information which has been determined to have a weak periodicity (a type including adventitious sounds) on the basis of an envelope, the information analyzing apparatus can clarify to a user whether or not such body information is classified as continuous adventitious sounds.

The waveform feature determining means may determine, in accordance with waveform feature determination criteria concerning the number of impulse noise components, whether or not a sound waveform contains a certain number or more of impulse noise components. If it is determined that the sound waveform contains the certain number or more of impulse noise components, the sound-type determining means may determine that there is a possibility that the body sound information belongs to discontinuous adventitious sounds.

If the number of impulse noise components contained in a sound waveform of body sound information (breath sounds) collected by a stethoscope is a certain number or more, it may mean that many instantaneous adventitious sounds (bursting sounds) other than expiration sounds and inspiration sounds are being generated. Accordingly, on the basis of a feature concerning the frequency occurrence of bursting sounds (the number of impulse noise components), the sound-type determining means can classify body sound information containing a certain number or more of impulse noise components as discontinuous adventitious sounds.

With this configuration, concerning body sound information which has been determined to have a weak periodicity (a type including adventitious sounds) on the basis of the number of impulse noise components, the information analyzing apparatus can clarify to a user whether or not such body information is classified as discontinuous adventitious sounds.

If a time for which the amplitude of the above-described envelope exceeds the amplitude average value continues for 200 ms or longer, the waveform feature determining means may determine, in accordance with the waveform feature determination criteria, that the envelope continues with the certain or greater value of amplitude for the certain period or longer.

Alternatively, if a total time for which the amplitude exceeds the amplitude average value in the envelope within a predetermined period is 200 ms or longer, the waveform feature determining means may determine, in accordance with the waveform feature determination criteria, that the envelope continues with the certain or greater value of amplitude for the certain period or longer.

If the waveform contains ten or more impulse noise components per period, the waveform feature determining means may determine, in accordance with the waveform feature determination criteria, that the sound waveform contains the certain number or more of impulse noise components.

The waveform feature determining means may determine, in accordance with waveform feature determination criteria concerning a frequency component distribution, whether or not a frequency component distribution of the sound waveform indicates that the sound waveform is likely to be normal or abnormal. If it is determined that the frequency component distribution of the sound waveform indicates that the sound waveform is likely to be normal, the sound-type determining means may determine that there is a possibility that the body sound information belongs to at least one of normal breath sounds and decreased breath sounds. If it is determined that the frequency component distribution of the sound waveform indicates that the sound waveform is likely to be abnormal, the sound-type determining means may determine that there is a possibility that the body sound information belongs to at least one of continuous adventitious sounds and discontinuous adventitious sounds.

If the frequency component distribution of body sound information (breath sounds) collected by a stethoscope is similar to a normal distribution, that is, if the sound waveform is likely to normal, it may mean that unwanted adventitious sounds other than expiration sounds and inspiration sounds are not contained. Accordingly, on the basis of a feature concerning the frequency component distribution of the sound waveform, the sound-type determining means can broadly classify the type of body sound information as a sound type without adventitious sounds (such as normal breath sounds and decreased breath sounds). On the other hand, if the frequency component distribution of the body sound information (breath sounds) is similar to an abnormal distribution, that is, if the sound waveform is likely to abnormal, it may mean that unwanted adventitious sounds other than expiration sounds and inspiration sounds are contained. Accordingly, on the basis of a feature concerning the frequency component distribution of the sound waveform, the sound-type determining means can broadly classify the type of body sound information as a sound type with adventitious sounds (such as continuous adventitious sounds and discontinuous adventitious sounds).

The waveform feature determining means may determine, in accordance with the waveform feature determination criteria concerning the frequency component distribution, that the frequency component distribution of the sound waveform indicates that the sound waveform is likely to be normal if total frequency components at 200 Hz or lower occupies 80% or higher of all frequency components in the frequency component distribution. The waveform feature determining means may determine, in accordance with the waveform feature determination criteria concerning the frequency component distribution, that the frequency component distribution of the sound waveform indicates that the sound waveform is likely to be abnormal if total frequency components at 200 Hz or higher occupies 30% or higher of all the frequency components in the frequency component distribution.

The waveform feature determining means may determine whether or not a periodicity of the sound waveform is strong, in accordance with waveform feature determination criteria for determining whether or not a periodicity of a sound waveform is strong. If it is determined that the periodicity of the sound waveform is strong, the sound-type determining means may determine that there is a possibility that the body sound information belongs to at least one of normal breath sounds and decreased breath sounds. If it is determined that the periodicity of the sound waveform is weak, the sound-type determining means may determine that there is a possibility that the body sound information belongs to at least one of continuous adventitious sounds and discontinuous adventitious sounds.

If a strong periodicity is observed in body sound information (breath sounds) collected by a stethoscope, it may mean that a cycle of expiration sounds and inspiration sounds is clear and unwanted adventitious sounds are not contained between expiration sounds and inspiration sounds. Accordingly, on the basis of a feature concerning the periodicity of the sound waveform, the sound-type determining means can broadly classify the type of body sound information into a sound type without adventitious sounds (such as normal breath sounds and decreased breath sounds) and a sound type with adventitious sounds (such as continuous adventitious sounds and discontinuous adventitious sounds).

The waveform feature determining means may determine, in accordance with waveform feature determination criteria concerning a frequency component distribution based on time-frequency analysis, whether or not there is a periodicity in each frequency range of the sound waveform. If it is determined that there is a periodicity in a high frequency range in the frequency component distribution based on time-frequency analysis, the sound-type determining means may determine that there is a possibility that the body sound information belongs to normal breath sounds. If it is determined that there is a periodicity in a low frequency range and there is no periodicity in a high frequency range in the frequency component distribution based on time-frequency analysis, the sound-type determining means may determine that there is a possibility that the body sound information belongs to decreased breath sounds.

If there is a periodicity in a high frequency range in a frequency component distribution based on time-frequency analysis conducted on body sound information (breath sounds) collected by a stethoscope, it may mean that there is no obstacle which blocks sounds (in particular, sounds in a high frequency range) in a path from lungs in which normal breath sounds are generated until a stethoscope. Accordingly, on the basis of a feature concerning the frequency component distribution based on time-frequency analysis conducted on the sound waveform, the sound-type determining means can further classify body sound information which has been determined to have a strong periodicity (sound type without adventitious sounds) as normal breath sounds.

In contrast, if a periodicity observed in a low frequency range is no longer observed (weakened) in a high frequency range, it may mean that there is an obstacle, such as pleural effusion, which blocks sounds (in particular, sounds in a high frequency range) in a path from lungs in which normal breath sounds are generated until a stethoscope. Accordingly, on the basis of a feature concerning the frequency component distribution based on time-frequency analysis conducted on the sound waveform, the sound-type determining means can further classify body sound information which has been determined to have a strong periodicity (sound type without adventitious sounds) as decreased breath sounds.

On the basis of a feature concerning the frequency component distribution based on time-frequency analysis, the information analyzing apparatus is able to clarity to a user whether body sound information which has been determined to have a strong periodicity (sound type without adventitious sounds) is normal breath sounds or decreased breath sounds.

The waveform feature determining means may determine, in accordance with the waveform feature determination criteria, that the periodicity of the sound waveform is strong if an autocorrelation function of the sound waveform has peaks at intervals of two to five seconds and if, in an envelope of the autocorrelation function, duration of a peak of the envelope with respect to a certain amplitude value is 10% or smaller of a breathing period.

The waveform feature determining means may determine, in accordance with the waveform feature determination criteria, that there is a periodicity in a high frequency range if there is a periodicity in a frequency range at 400 Hz or higher in the frequency component distribution of the sound waveform based on time-frequency analysis. The waveform feature determining means may determine, in accordance with the waveform feature determination criteria, that there is a periodicity in a low frequency range and there is no periodicity in a high frequency range if a frequency at which a periodicity is observed is a frequency range lower than 400 Hz in the frequency component distribution of the sound waveform based on time-frequency analysis.

The information analyzing apparatus may further include abnormality-level determining means for determining, if the sound-type determining means determines that there is a possibility that the body sound information belongs to abnormal sounds, a degree of abnormality of the abnormal sounds on the basis of a feature of the sound waveform specified by the waveform feature determining means.

With this configuration, the information analyzing apparatus is able to clarify to a user, not only the sound type of body sound information, but also, if body sound information is abnormal, the degree (level) of the abnormality.

The sound-type determining means may determine whether or not the body sound information matches each of predefined sound types.

If the predefined sound types are, for example, the above-described normal breath sounds, decreased breath sounds, continuous adventitious sounds, and discontinuous adventitious sounds, though they are not restricted thereto, the information analyzing apparatus is able to clarify to a user whether or not the body sound information matches normal breath sounds, whether or not the body sound information matches decreased breath sounds, whether or not the body sound information matches continuous adventitious sounds, and whether or not the body sound information matches discontinuous adventitious sounds.

Alternatively, the sound-type determining means may specify which any one of a plurality of predefined sound types that the body sound information matches.

If the predefined sound types are, for example, the above-described normal breath sounds, decreased breath sounds, continuous adventitious sounds, and discontinuous adventitious sounds, though they are not restricted thereto, the information analyzing apparatus is able to clarify to a user which any one of the normal breath sounds, decreased breath sounds, continuous adventitious sounds, and discontinuous adventitious sounds the body sound information matches.

The information analyzing apparatus may further include result output means for outputting sound-type determination results that indicate a sound type to which the body sound information belongs and that are generated by the sound-type determining means to a display unit.

The result output means may associate the sound-type determination results with the body sound information and store the sound-type determination results in a storage unit.

The above-described information analyzing apparatus of the present invention may be mounted on a digital stethoscope. In this case, the digital stethoscope serves as the information analyzing apparatus of the present invention.

In order to solve the above-described problems, an information analyzing method of the present invention includes: a waveform feature determining step of applying waveform feature determination criteria to a sound waveform included in body sound information collected by a stethoscope so as to specify a feature of the sound waveform, the waveform feature determination criteria indicating criteria for classifying features of sound waveforms; and a sound-type determining step of determining a sound type to which the body sound information belongs, on the basis of the feature of the sound waveform specified in the waveform feature determining step.

In order to solve the above-described problems, a measurement system according to one mode of the present invention includes: a digital stethoscope for conducting auscultation on a subject; the above-described any one of information analyzing apparatuses that analyze body sound information collected by the digital stethoscope; and an imaging apparatus that performs imaging processing on the subject on the basis of auscultation results obtained by conducting auscultation by using the digital stethoscope and output from the information analyzing apparatus. The imaging apparatus includes auscultation-result obtaining means for obtaining auscultation results which at least include information concerning the presence or the absence of abnormality determined by the information analyzing apparatus on the basis of the body sound information and information concerning a part from which the body sound information has been collected, part specifying means for specifying a part for which the occurrence of abnormality has been determined, on the basis of the auscultation results obtained by the auscultation-result obtaining means, and imaging control means for performing imaging on a part specified by the part specifying means in a manner different from a manner for other parts so as to obtain image data concerning the subject.

With this configuration, the imaging apparatus is able to perform imaging processing by utilizing auscultation results output from the information analyzing apparatus. That is, the cooperation between measurements of auscultation sounds and imaging can be implemented. For example, imaging can be performed by focusing on a specific part in which an abnormality is observed in body sound information. Additionally, if there is no problem for a certain part in the results of auscultation sounds, a situation in which the imaging operation is uselessly performed for this part can be avoided.

The information analyzing apparatus may be implemented by a computer. In this case, a control program for the information analyzing apparatus which implements the information analyzing apparatus by using a computer as a result of operating the computer as each of the above-described means is also encompassed within the present invention. A computer-readable recording medium on which the control program is recorded is also encompassed within the present invention.

### INDUSTRIAL APPLICABILITY

An information analyzing apparatus of the present invention is able to perform information processing on body sound information measured and collected by a stethoscope and to determine a sound type of body sound on the basis of features of the body sounds. Accordingly, the information analyzing apparatus of the present invention can be widely used in a system in which the condition of a living body emitting body sounds is determined by using information concerning these body sounds. In particular, the information analyzing apparatus of the present invention is suitably used in an auscultation system in which the condition of a patient is determined and diagnosis and treatment is conducted for the patient by using collected body sound information.

### REFERENCE SIGNS LIST

1 clinic
2 support center (remote site)
3 digital stethoscope (stethoscope)
4 management server
5 communication network
10 controller
11 input unit
12 display unit
13 storage unit
14 communication unit
20 body sound obtaining unit (body sound obtaining means)
21 body sound processor (body sound processing means)
22 body sound analyzer (body sound analyzing means)
23 result output unit (result output means)
30 waveform feature determining unit (waveform feature determining means)
31 periodicity determining section (waveform feature determining means/periodicity determining means)
32 spectrum determining section (waveform feature determining means/frequency component distribution determining means)
33 spectrogram determining section (waveform feature determining means/frequency-range periodicity determining means)
34 envelope determining section (waveform feature determining means/envelope determining means)
35 impulse noise determining section (waveform feature determining means/impulse noise determining means)
40 sound-type determining unit (sound-type determining means)
41 normal-breath-sound determining section (sound-type determining means/normal-breath-sound determining means)
42 decreased-breath-sound determining section (sound-type determining means/decreased-breath-sound determining means)
43 continuous-adventitious-sound determining section (sound-type determining means/continuous-adventitious-sound determining means)
44 discontinuous-adventitious-sound determining section (sound-type determining means/discontinuous-adventitious-sound determining means)
45 comprehensive determination section (sound-type determining means/comprehensive determination means)
50 abnormality-level determining unit (abnormality-level determining means)
51 decreased-sound-level determining section (abnormality-level determining means/decreased-sound-level determining means)
52 continuity-level determining section (abnormality-level determining means/continuity-level determining means)
53 discontinuity-level determining section (abnormality-level determining means/discontinuity-level determining means)
100 information analyzing apparatus
200 auscultation system
211 autocorrelation analyzer (body sound processing means)
212 Fourier transform unit (body sound processing means)
213 time-frequency analyzer (body sound processing means)
214 envelope detector (body sound processing means)
215 impulse noise detector (body sound processing means)
3006 imaging apparatus
3010 controller
3011 communication unit
3012 storage unit
3013 imaging unit
3020 auscultation-result obtaining section (auscultation-result obtaining means)
3021 imaging-part specifying section (part specifying means)
3022 imaging control section (imaging control means)
3600 measurement system

1-24. (canceled)

25. An information analyzing apparatus comprising:
waveform feature determining means for applying waveform feature determination criteria to a sound waveform included in body sound information collected by a stethoscope so as to specify a feature of the sound waveform, the waveform feature determination criteria indicating criteria for classifying features of sound waveforms; and
sound-type determining means for determining a sound type to which the body sound information belongs, on the basis of the feature of the sound waveform specified by the waveform feature determining means,
wherein the waveform feature determining means makes a determination of
(i) whether or not an envelope of the sound waveform continues with a certain or greater value of amplitude, in accordance with of waveform feature determination criteria concerning an envelope,
(ii) whether or not the sound waveform contains a certain number or more of impulse noise components, in accordance with waveform feature determination criteria concerning the number of impulse noise components,
(iii) whether or not a frequency component distribution of the sound waveform indicates that the sound waveform is likely to be normal or abnormal, in accordance with waveform feature determination criteria concerning a frequency component distribution,
(iv) whether or not a periodicity of the sound waveform is strong, in accordance with waveform feature determination criteria for determining whether or not a periodicity of a sound waveform is strong, or
(v) whether or not there is a periodicity in each frequency range of the sound waveform, in accordance with waveform feature determination criteria concerning a frequency component distribution based on time-frequency analysis.

26. The information analyzing apparatus according to claim 25, wherein:
the waveform feature determination criteria include a threshold to be compared with a feature quantity found from the sound waveform and a condition determined by the threshold; and
the waveform feature determining means specifies a feature of the sound waveform by determining whether or not the feature quantity of the sound waveform matches the condition.

27. The information analyzing apparatus according to claim 25, wherein a sound type to be determined by the sound-type determining means is at least one of
“normal breath sounds” indicating that breath sounds emitted from a living body are normal,
“decreased breath sounds” indicating that breath sounds emitted from a living body are decreased before the breath sounds are collected by a stethoscope,
“continuous adventitious sounds” indicating that breath sounds emitted from a living body include continuous adventitious sounds, and
“discontinuous adventitious sounds” indicating that breath sounds emitted from a living body include discontinuous adventitious sounds.

28. The information analyzing apparatus according to claim 27, wherein:
if it is determined that the envelope continues with the certain or greater value of amplitude, the sound-type determining means determines that there is a possibility that the body sound information belongs to “continuous adventitious sounds”.

29. The information analyzing apparatus according to claim 27, wherein:
if it is determined that the sound waveform contains the certain number or more of impulse noise components, the sound-type determining means determines that there is a possibility that the body sound information belongs to “discontinuous adventitious sounds”.

30. The information analyzing apparatus according to claim 25, wherein, if a time for which the amplitude of the envelope exceeds an amplitude average value continues for 200 ms or longer, the waveform feature determining means determines, in accordance with the waveform feature determination criteria, that the envelope continues with the certain or greater value of amplitude.

31. The information analyzing apparatus according to claim 25, wherein, if a total time for which the amplitude exceeds an amplitude average value in the envelope within a predetermined period is 200 ms or longer, the waveform feature determining means determines, in accordance with the waveform feature determination criteria, that the envelope continues with the certain or greater value of amplitude.

32. The information analyzing apparatus according to claim 25, wherein, if the waveform contains ten or more impulse noise components per period, the waveform feature determining means determines, in accordance with the waveform feature determination criteria, that the sound waveform contains the certain number or more of impulse noise components.

33. The information analyzing apparatus according to claim 27, wherein:
if it is determined that the frequency component distribution of the sound waveform indicates that the sound waveform is likely to be normal, the sound-type determining means determines that there is a possibility that the body sound information belongs to at least one of “normal breath sounds” and “decreased breath sounds”; and
if it is determined that the frequency component distribution of the sound waveform indicates that the sound waveform is likely to be abnormal, the sound-type determining means determines that there is a possibility that the body sound information belongs to at least one of “continuous adventitious sounds” and “discontinuous adventitious sounds”.

34. The information analyzing apparatus according to claim 25, wherein:
the waveform feature determining means determines, in accordance with the waveform feature determination criteria concerning the frequency component distribution, that the frequency component distribution of the sound waveform indicates that the sound waveform is likely to be normal if total frequency components at 200 Hz or lower occupies 80% or higher of all frequency components in the frequency component distribution; and
the waveform feature determining means determines, in accordance with the waveform feature determination criteria concerning the frequency component distribution, that the frequency component distribution of the sound waveform indicates that the sound waveform is likely to be abnormal if total frequency components at 200 Hz or higher occupies 30% or higher of all the frequency components in the frequency component distribution.

35. The information analyzing apparatus according to claim 27, wherein:
if it is determined that the periodicity of the sound waveform is strong, the sound-type determining means determines that there is a possibility that the body sound information belongs to at least one of “normal breath sounds” and “decreased breath sounds”; and
if it is determined that the periodicity of the sound waveform is weak, the sound-type determining means determines that there is a possibility that the body sound information belongs to at least one of “continuous adventitious sounds” and “discontinuous adventitious sounds”.

36. The information analyzing apparatus according to claim 27, wherein:
if it is determined that there is a periodicity in a high frequency range in the frequency component distribution based on time-frequency analysis, the sound-type determining means determines that there is a possibility that the body sound information belongs to “normal breath sounds”; and
if it is determined that there is a periodicity in a low frequency range and there is no periodicity in a high frequency range in the frequency component distribution based on time-frequency analysis, the sound-type determining means determines that there is a possibility that the body sound information belongs to “decreased breath sounds”.

37. The information analyzing apparatus according to claim 25, wherein the waveform feature determining means determines, in accordance with the waveform feature determination criteria, that the periodicity of the sound waveform is strong if an autocorrelation function of the sound waveform has peaks at intervals of two to five seconds and if, in an envelope of the autocorrelation function, duration of a peak of the envelope with respect to a certain amplitude value is 10% or smaller of a breathing period.

38. The information analyzing apparatus according to claim 25, wherein:
the waveform feature determining means determines, in accordance with the waveform feature determination criteria, that there is a periodicity in a high frequency range if there is a periodicity in a frequency range at 400 Hz or higher in the frequency component distribution of the sound waveform based on time-frequency analysis; and
the waveform feature determining means determines, in accordance with the waveform feature determination criteria, that there is a periodicity in a low frequency range and there is no periodicity in a high frequency range if a frequency at which a periodicity is observed is a frequency range lower than 400 Hz in the frequency component distribution of the sound waveform based on time-frequency analysis.

39. The information analyzing apparatus according to claim 25, further comprising:
abnormality-level determining means for determining, if the sound-type determining means determines that there is a possibility that the body sound information belongs to abnormal sounds, a degree of abnormality of the abnormal sounds on the basis of a feature of the sound waveform specified by the waveform feature determining means.

40. The information analyzing apparatus according to claim 25, wherein the sound-type determining means determines whether or not the body sound information matches each of predefined sound types.

41. The information analyzing apparatus according to claim 25, wherein the sound-type determining means specifies which any one of a plurality of predefined sound types that the body sound information matches.

42. A digital stethoscope comprising:
the information analyzing apparatus according to claim 25.

43. A control method for an information analyzing apparatus, comprising:
a waveform feature determining step of applying waveform feature determination criteria to a sound waveform included in body sound information collected by a stethoscope so as to specify a feature of the sound waveform, the waveform feature determination criteria indicating criteria for classifying features of sound waveforms; and
a sound-type determining step of determining a sound type to which the body sound information belongs, on the basis of the feature of the sound waveform specified in the waveform feature determining step,
wherein the waveform feature determining step makes a determination of
(i) whether or not an envelope of the sound waveform continues with a certain or greater value of amplitude, in accordance with of waveform feature determination criteria concerning an envelope,
(ii) whether or not the sound waveform contains a certain number or more of impulse noise components, in accordance with waveform feature determination criteria concerning the number of impulse noise components,
(iii) whether or not a frequency component distribution of the sound waveform indicates that the sound waveform is likely to be normal or abnormal, in accordance with waveform feature determination criteria concerning a frequency component distribution,
(iv) whether or not a periodicity of the sound waveform is strong, in accordance with waveform feature determination criteria for determining whether or not a periodicity of a sound waveform is strong, or
(v) whether or not there is a periodicity in each frequency range of the sound waveform, in accordance with waveform feature determination criteria concerning a frequency component distribution based on time-frequency analysis.

44. A measurement system comprising:
a digital stethoscope for conducting auscultation on a subject;
the information analyzing apparatus that analyzes body sound information collected by the digital stethoscope; and
an imaging apparatus that performs imaging processing on the subject on the basis of auscultation results obtained by conducting auscultation by using the digital stethoscope and output from the information analyzing apparatus,
the information analyzing apparatus including
waveform feature determining means for applying waveform feature determination criteria to a sound waveform included in body sound information collected by a stethoscope so as to specify a feature of the sound waveform, the waveform feature determination criteria indicating criteria for classifying features of sound waveforms; and
sound-type determining means for determining a sound type to which the body sound information belongs, on the basis of the feature of the sound waveform specified by the waveform feature determining means,
the imaging apparatus including
auscultation-result obtaining means for obtaining auscultation results which at least include information concerning the presence or the absence of abnormality determined by the information analyzing apparatus on the basis of the body sound information and information concerning a part from which the body sound information has been collected,
part specifying means for specifying a part for which the occurrence of abnormality has been determined, on the basis of the auscultation results obtained by the auscultation-result obtaining means, and
imaging control means for performing imaging on a part specified by the part specifying means in a manner different from a manner for other parts so as to obtain image data concerning the subject.
